(19) United States
(12) Patent Application Publication
(10) Pub. No.: US 2009/0305955 A1
(43) Pub. Date: Dec. 10, 2009

(54) Cyclopeptide with Anti-Cancer Activity Derived from Collagen Type IV

(76) Inventors: Jean-Claude Paul Louis Monboisse, Reims (FR); Laurent Gabriel Emile Ramont, Lavannes (FR); Sylvie Anne Yvonne Brassart, Reims (FR); Nicolas Joseph Gilbert FLoquet, Reims (FR); Jessica Andree Mireille Thevenard, Reims (FR)

Correspondence Address: BIRCH STEWART KOLASCH & BIRCH, PO BOX 747, FALLS CHURCH, VA, 22040-0747, US

(21) Appl. No.: 12/225,419

(22) PCT Filed: Mar. 22, 2007
(86) PCT No.: PCT/FR2007/000487

§ 371 Date: May 5, 2009

(30) Foreign Application Priority Data
Mar. 23, 2006 (FR) 06/2530

(51) Int. Cl. A61K 38/12; C07K 7/64; A61P 35/00

(52) U.S. Cl. 514/9; 530/317

(57) ABSTRACT

The present invention relates to a cyclopeptide characterized in that it comprises the YSNS amino acid sequence, and more particularly a cyclopentapeptide which forms a β-bend structure at the YSNS amino acids. In one specific embodiment, the cyclopeptide of the invention is capable of binding to the αVβ3-integrin. The application also claims the use of a cyclopeptide of the invention in the treatment of cancer, and more particularly in the treatment of the various forms of melanoma, and also in the manufacture of a medicament for treating cancer. Finally, the application describes the use of a cyclopeptide of the invention for inhibiting or reducing angiogenesis, and more particularly in tumours, and also in the manufacture of a medicament for inhibiting or reducing angiogenesis.

The invention concerns a cyclopeptide characterized in that it comprises the amino acid sequence YSNS (SEQ ID NO: 1). In particular, the cyclopeptide is a pentapeptide forming a β-bend structure at the YSNS (SEQ ID NO: 1) amino acids and which is capable of being obtained by forming a peptide bond between the two amino acids at the ends of the pentapeptide represented linearly. In a preferred embodiment, the cyclopeptide of the invention is capable of binding to αVβ3 integrin.

The use of a cyclopeptide as defined above in the treatment of cancer, and more particularly in the treatment of the various forms (or stages) of melanoma, and in the manufacture of a drug for the treatment of cancer, also form part of the invention. The cyclopeptide is particularly suitable for oral administration of a composition containing it.

Melanoma holds second place for cancer in man in terms of the numbers of years of life lost. During the last ten years, its incidence has increased more (3% to 5% per annum) than other types of cancer with the exception of bronchial cancer in women. In 2000, it was estimated that one individual in 100 would develop a melanoma during his lifetime and that one patient in two would not get to 50 years of age. That type of cancer has inevitably become a major problem to public health. Melanoma is a malignant tumour with a very poor prognosis and a high risk of visceral and ganglionic metastases.

Over the last few years, the tumour mechanism has been studied and the mechanisms underlying tumour progression have gradually been elucidated. Thus, during tumour progression, cancer cells leave the primary tumour, cross vascular membranes and migrate into the surrounding extracellular matrix.

This set of phenomena involves secretion then activation of proteolytic enzymes, such as matrix metalloproteinases (MMPs) or the plasminogen activation system (Hornebeck W et al). The expression profile of said proteolytic enzymes, studied in various human or mouse melanoma cell lines, have revealed a substantial increase in the expression of various MMPs in a manner which correlates with the invasive phenotype of those cells (Egeblad M et al). Expression of functionally active MMP-2 at the cell surface directly influences adhesion and spreading of cells onto components of the extracellular matrix and basal membranes, and encourages migration and invasion of said cells.

The activation of MMP-2 requires the formation of complexes with its inhibitor, TIMP-2, and another transmembrane MMP, MT1-MMP or MMP-14 (Egeblad M et al). It also requires the presence of αVβ3 integrin the expression of which increases with the degree of invasivity of the melanoma cells. This integrin acts as a membrane receptor for MMP-2, encourages its activation and focuses the active form of MMP-2 into the lamellipods, which induce progression of the cancer cell into the extracellular matrix (Settor R E B et al; Deryugina E I et al). Further, during tumour progression, a number of inflammatory phenomena involving the activation of inflammatory cells intervene (polynuclear nucleophiles, monocytes, macrophages, etc) which result in the release of various cytokines and growth factors, but also MMPs, such as MMP-9.

The increase in tumour volume causes hypoxia and a deficit in the supply of nutrients for the tumour cells. The effects are overcome by tumoral angiogenesis, a neovascularization mechanism occurring in tumours from a pre-existing capillary network. It is vital for the growth of tumours and the development of metastases as it re-establishes the supply of oxygen and nutrients. The activation of endothelial cells, induced by hypoxia and proto-oncogens within the tumours, leads to degradation of the basal membrane and the surrounding extracellular matrix by means of the proteolytic cascades involved in tumour progression. Orientated migration of the endothelial cells is followed by a proliferative phase. Cells organize themselves into a capillary type structure to form the intratumoral vascular network. Angiogenesis is a prognostic factor in various cancers including melanoma.

The sole role of mechanical support was long ago put down to macromolecules of the extracellular matrix. A few years ago, it was shown that certain constituent protein domains of such macromolecules could also control various physiopathological events such as cell differentiation, apoptosis or gene expression. Matrikines are peptides derived from partial protolysis of macromolecules of the extracellular matrix which are capable of regulating the biological activity of various cell types (Maquart F X et al). Molecules of the extracellular matrix, more particularly the various constituents of the basal membranes (type IV, XV and XVIII collagens, laminins, proteoglycans, etc) may regulate the adhesion and migration of cancer cells via certain matrikines (Pasco S et al, 2004); Ortega N et al). Similarly, these latter may ensure control of the expression and/or activation of proteases employed during tumour progression.

Among the macromolecules of the basal membranes, type IV collagen, which is the main constituent, plays a dominant role in said control via the intermediary of the non collagenic domain (NC1) of its various chains. It is constituted by the triple helix association of three polypeptide chains α(IV) out of 6 possible chains, α1(IV) to α6(IV), each coded by a different gene (Kalluri R). The most frequent association corresponds to [α1(IV)2; α2(IV)]; it is encountered in all basal membranes. Other associations, which are less well determined, contain the α3(IV) to α6(IV) chains, termed minor chains because of their lesser expression. The α3(IV) chain has a highly specialized tissue distribution (pulmonary alveolus, renal glomerule, crystalline capsule, etc) (Kalluri R). Matrikines derived from the NC1 domains of the α(IV) chains of type IV collagen or from their helical domain induce adhesion of cancer cells and control their invasive properties (Pasco S et al, 2004; Ortega N et al; Kalluri R).

The preceding studies have focussed on the C-terminal domain of the α3(IV) chain (amino acids 1438 to 1670 of sequence NP_{—}000082; NCBI accession number) termed the NC1 (noncollagenous domain) which has proved to be extremely interesting in the light of the following results:
the NC1 domain of the α3(IV) chain has an inhibiting activity on the proliferation and invasive properties of various cancer cell lines. More particularly, a peptide constituted by amino acids 185 to 203 of the NC1 α3(IV) domain (CNYYSNSYSFWLASLNPER) (SEQ ID NO: 6) may inhibit the proliferation of said various cell lines. In contrast, the peptides constituted by homologous sequences of the other α(IV) chains have no effect on those same lines (Han J et al; Shahan S et al).
further, peptides of the [NC1 α3(IV) 185-203] sequence inhibit the migration of melanoma or fibrosarcoma cells in vitro. This inhibition of the migration of tumour cells appears to be explained by the inhibiting effect of the [NC1 α3(IV) 185-203] sequence on binding of MMP-2 to the plasma membrane but also by the inhibiting effect on the activation of MMP-2 (Pasco S et al, 2000a). Comparable results have been obtained with bronchial cancer cells (Martinella-Catusse et al, 2001);
the [NC1 α3(IV) 185-203] sequence binds to the αVβ3 integrin-CD47 protein complex present on the surface of melanoma cells (Shahan T A et al). More precisely, the [NC1 α3(IV) 185-203] sequence binds to the β3 sub-unit of the integrin, independently of CD47 and of the associated a sub-unit (Pasco S et al, 2000b). Similarly, the interaction domain between that sequence and the β3 sub-unit is independent of the recognition site of the RGD sequence, which acts as a recognition site for many proteins of the extracellular matrix on various integrins;
the [NC1 α3(IV) 185-203] synthetic peptide inhibits the tumoral growth of murine melanoma B16F1 cells, pre-incubated with the [NC1 α3(IV) 185-203] synthetic peptide, then injected into C57BL6 syngenic mice; this inhibition is exerted by a reduction in the proliferation of melanoma cells and of their invasive properties and by a reduction in the expression of MMP-2 and the plasminogen activators u-PA and t-PA. This inhibiting effect depends on the structural conformation of the [NC1 α3(IV) 185-203] sequence which forms a β-bend at the amino acids YSNS (SEQ ID NO: 1) (188-191) which is vital to its biological activity. The absence of this β-bend in analogous structures abolishes the biological activity (Floquet et al);
a peptide of the α3(IV) 185-203 sequence is capable of inhibiting angiogenesis in vitro and in vivo, as shown on histological sections produced from tumours treated by that peptide and in which the number of blood vessels is substantially reduced (Pasco S et al, 2005);
the inhibiting activity of the α3(IV) 185-203 sequence on tumour growth of melanoma cells may be reproduced using a linear heptapeptide containing the 7 N-terminal amino acids CNYYSNS (SEQ ID NO: 5) in the domain, including the YSNS (SEQ ID NO: 1) sequence. Structural studies have shown the formation of a β-bend structure at the tetrapeptide YSNS (SEQ ID NO: 1), which is vital to biological activity. Homologous peptides not having this particular conformation are deprived of biological activity (Floquet et al).

Despite elucidation of those various mechanisms involved in the initiation and evolution of melanomas, only a few treatments have been proposed and they are limited in their effectiveness. Hence, surgical excision is currently the only curative treatment for stage 1 melanoma. It must be early and extensive. At more advanced stages, and in particular after metastatic dissemination, current palliative treatments are disappointing and of little effect. They are primarily down to polychemotherapy and little progress has been made.

The present invention proposes molecules and more particularly molecules produced from peptides derived from type IV collagen having anti-tumoral properties, and which satisfy the demands imposed for their therapeutic use, namely good solubility and bioavailability and effective biological activity.

The invention concerns a cyclopeptide, characterized in that it comprises the sequence of amino acids YSNS (SEQ ID NO: 1) or in that it consists in this sequence. In a particular embodiment of the invention, this cyclopeptide is capable of binding to αVβ3 integrin.

The term “cyclopeptide” means a molecule formed by a sequence of amino acid residues (peptide) which exists with at least one stable bond between two of its residues, allowing the formation of a cycle constituted by all of the amino acid residues or by a portion of the amino acid sequence, said portion comprising the consecutive residues YSNS (SEQ ID NO: 1), either bonded via peptide bonds or at least two thereof by said stable bond. Said stable bond thus closes the cycle at the two residues of the amino acid sequence. Alternatively, the expression “cyclic peptide” is also used, as opposed to the terms “linear peptide” or “acyclic peptide”. In the context of the invention, two types of cyclopeptides are encompassed in the term “cyclopeptide”:

homodetic cyclopeptides, which consist solely of amino acid residues bonded to each other via peptide bond or eupeptide bond (a peptide bond between the alpha-carboxyl function of one amino acid and the alpha-amino function of another amino acid); and

heterodetic cyclopeptides, which consist of amino acid residues bonded to each other via peptide bonds and at least one bond of another nature such as an ester bond, a disulphide bridge, a carbon-carbon bond, a carbon-nitrogen bond, a nitrogen-hydrogen bond or a carbon-sulphur bond. In this case, the bond may be between the N- and C-terminal amino acid functions, between the function of a terminal amino acid and the function of a side chain (internal amino acid), or between two side chains.

In a particular embodiment, the invention concerns a homodetic cyclopeptide comprising the sequence YSNS (SEQ ID NO: 1), i.e. the cycle is produced by cyclisation of the amine group and carboxyl group of the N- and C-terminal amino acids, thereby forming a cyclic peptide linked via an amide bond.

The cyclopeptides of the invention, or in a particular embodiment the cycle of said cyclopeptides, are at least 4 amino acids in size, for example less than 10 amino acids in size, and in particular with a number of amino acid residues from 4 to 6, and more particularly exactly 4 (cyclotetrapeptide), exactly 5 (cyclopentapeptide) or exactly 6 (cyclohexapeptide).

In a particular embodiment, the cyclopeptide of the invention does not consist of a cyclopeptide with sequence ACPYSNSSLC (SEQ ID NO: 11).

As an example, a heterodetic cyclopentapeptide of the invention will be represented by the formula:

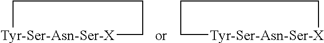

in which a horizontal line to the side of the amino acid indicates a bond with the amine or carboxyl function (normally engaged in the peptide bond), and a vertical line above the amino acid indicates a bond with a group other than that the ones indicated above.

By way of example, a homodetic cyclopentapeptide is represented by any one of the following formulae:

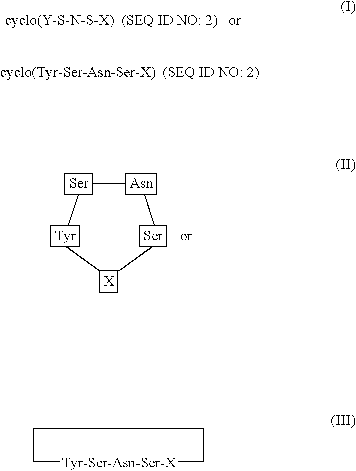

Among the functional properties of the cyclopeptides of the invention illustrating their use in the production of antitumoral compositions, in a particular embodiment thereof, binding to αVβ3 integrin is concerned. In a particular embodiment, the cyclopeptides bind to αVβ3 integrin with at least as much effectiveness as the linear peptide with sequence [NC1 α3(IV) 185-203] and/or the linear peptide CNYYSNS (SEQ ID NO: 5). More particularly still, binding of the cyclopeptide to αVβ3 integrin is more effective than the linear peptides described above.

Binding studies on the linear heptapeptide CNYYSNS (SEQ ID NO: 5) or the cyclopeptide YSNSG (SEQ ID NO: 3) of the invention were carried out by competition with the native linear peptide [NC1 α3(IV) 185-203] labelled with biotin (Pasco et al, 2000b). Briefly, melanoma cells, for example UACC-903, HT-144 or A-375 (10⁶ cells) were pre-incubated in vitro for 15 min in the presence of the linear heptapeptide CNYYSNS (SEQ ID NO: 5) (0 to 200 μM) or the cyclopeptide YSNSG (SEQ ID NO: 3) (0 to 200 μM). The cells were washed 3 times, then incubated for 30 min with the peptide [NC1 α3(IV) 185-203] labelled with biotin. The cells were then washed 3 times and incubated with an anti-biotin monoclonal antibody labelled with FITC (fluorescein isothiocyanate) in 1:75 dilution. The cells were then fixed and analyzed by immunofluorescence.

Similarly, the potential regions of interaction of the cyclic peptide or cyclic peptides of the invention and/or their linear versions were characterized using docking methods. These methods consist in predicting the bonding mode and affinity of flexible ligands, knowing the structure of the target, which is considered to be rigid. The structure of αVβ3 integrin has been published. Using Autodock software, the docking study protocol consists in reading the structure of the target (available in the Protein Data Bank) then successively testing all the possible positions and orientations of the ligand at its surface. The positions are classified using a score function describing the free binding energy of the ligand to the target protein. The best positions are analyzed in terms of regions of high affinities to the surface of the receptor (clusters). The regions concerned are then verified experimentally by studying the interactions between the peptides of the invention and recombinant domains, mutated for the amino acids involved, of the β3 integrin, using a technique employing a Biacore apparatus.

The cyclopeptides of the invention, whether they are homodetic or heterodetic in particular, may also be characterized by the presence, in the amino acids YSNS (SEQ ID NO: 1), of a β-bend structure as shown in FIG. 1. In particular, the dihedral angles φ and ψ of the two central residues of the Y(SN)S bend are respectively approximately −90 and approximately −66 degrees. The corresponding β-bend is close to a type I β-bend. Thus, the β-bend formed by the amino acids YSNS (SEQ ID NO: 1) is of type I, type VIII (classification by Hutchinson E G et al) or similar to those β-bend types by dint of the dihedral angles mentioned above. In particular, the cyclopeptide of the invention has a β-bend with a value ψ+1 of about −60° and a value φ+2 of approximately −90°. The presence of a β-bend in one of the cyclopeptides of the invention requires that the peptide bonds between the amino acid residues Y₁ and S₂, S₂ and N₃, and N₃ and S₄ are in a trans orientation.

The cyclopeptides of the invention have anti-tumoral capacities illustrated by the fact that at least one of the following properties is observed: their capacity to inhibit the proliferation of tumour cells, their capacity to inhibit the migration of tumour cells, or an anti-angiogenic activity. A cyclopeptide is considered to have at least one of these inhibition properties if said inhibition of proliferation or cell migration or anti angiogenic activity is at least as effective as that recorded with the linear heptapeptide CNYYSNS (SEQ ID NO: 5).

The inhibition of the proliferation or migration of tumour cells may be tested as indicated in the section entitled “methods” in points A.9 and A.10 respectively. The experiments described in these paragraphs may be carried out with any type of tumour cells, and in particular melanoma tumour cells such as UACC-903 cells, HT-144 cells (ATCC HTB-63), A375 cells (ATCC CRL-1619) or G-361 cells (ATCC CRL-1424) (LGC Promochem-ATCC). Regarding the anti-angiogenic activity, it may be tested using the method described in point A.15 using endothelial cells such as HMEC-1 cells (Ades, Atlanta) or HUVEC cells (Promocell, Heidelberg, Germany).

Thus, a cyclopeptide in accordance with the invention is considered to be effective as regards the inhibition of the proliferation of tumour cells (and more particularly cells from melanomas) when, in the presence of said cyclopeptide, the percentage proliferation of cells observed in vitro and preferably in vivo is reduced by at least 20%, at least 30% or at least 40% with concentrations of cyclopeptide as low as 5 to 20 μM. Preferably, a reduction of 50% in the proliferation of tumour cells is achieved with a concentration of 20 μM for an initial number of 20,000 cells, per well.

Further, a cyclopeptide of the invention is considered to be effective as regards the inhibition of migration of tumour cells (and more particularly cells derived from melanoma) when the number of cells observed is reduced by a factor of at least 1.5 after in vitro and preferably in vivo observation. Preferably, a reduction by a factor of 2 is considered to be particularly effective at a concentration of 20 μM for an initial number of cells of 5×10⁴.

Finally, the anti-angiogenic activity was evaluated by the capacity of endothelial cells (HMEC-1 cells or HUVEC cells) to form capillary pseudotubes when cultivated on a Matrigel gel. A cyclopeptide in accordance with the invention is considered to have an anti-angiogenic activity when the percentage by number or size of the capillary pseudotubes is reduced by at least 40%, preferably at least 50% for a concentration of at least 10 μM of cyclopeptide.

In addition to the functional characteristics described above, the cyclopeptide of the invention is sufficiently bioavailable and/or stable to be used in pharmaceutical compositions. In particular, the cyclopeptide is as bioavailable and/or as stable as the linear heptapeptide CNYYSNS (SEQ ID NO: 5).

The term “bioavailable peptide” means a peptide which is capable of crossing the various biological barriers to reach target cells after its administration, and particularly to pass through the intestinal barrier after oral administration. Bioavailability is determined for a selected cell type as a function of the envisaged application.

The term “stable peptide” means a peptide which has a lifetime, once administered in vivo, which is sufficient to reach target cells and to exert its biological action. Such a peptide has a conformation which protects it against degradation by cell proteases while retaining its biological activity. An indication of the stability of a peptide may be obtained using tests carried out in vitro. In vitro degradation of a cyclopeptide is measured by contact with a variety of purified proteases, which are commercially available, for increasing incubation periods (1 hour to 72 hours, for example). Peptide degradation is then demonstrated by reverse phase HPLC, comparing the profiles obtained before and after digestion. The biological activity of peptides which undergo proteolytic degradation is verified by measuring the inhibition of migration of tumour cells using the described technique.

In a particular embodiment, the cyclopeptide is a tetrapeptide with sequence YSNS (SEQ ID NO: 1), which may be represented by the formula:

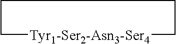

Cyclisation of the linear peptide YSNS (SEQ ID NO: 1) is facilitated by the proximity which exists between the Cα groups (see FIG. 1) of Y₁ and S₄, which are separated by less than 7 Å.

In another embodiment, the cyclopeptide is a cyclopentapeptide with sequence YSNSX (SEQ ID NO: 2) with the formula as defined above, and in which X is any amino acid which allows cyclisation of the linear peptide with the same sequence. The nature of the amino acid residue X is restricted by the formation of a cyclopeptide, which may be homodetic, and also by conservation of the β-bend in the YSNS (SEQ ID NO: 1) residues. Preferably, an amino acid is used with a very small volume (such as alanine, glycine or serine) or with a small volume (such as cysteine, asparagine or threonine) in which the side chains do not prevent the formation of peptide bonds between Y₁ and X₅ and between S₄ and X₅. Alternatively or in combination with the restriction noted above, an amino acid which has a small charge or is neutral is preferably used (such as alanine, asparagine, cysteine, glycine, serine or threonine) to avoid any undesirable interaction which would alter the conformation of the β-bend. Particular examples of these amino acids are alanine and glycine.

Thus, a cyclopeptide in accordance with the invention is a cyclopeptide, which may, be homodetic or heterodetic, consisting of the sequence YSNSG (SEQ ID NO: 3). Preferably, the cyclopeptide is a homodetic pentacyclopeptide wherein the 5 amino acids are linked by peptide linkages, with formula:

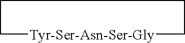

or with formula:

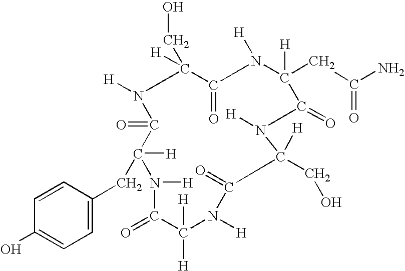

In one embodiment of the invention, the homodetic cyclopeptide (regardless of size) has trans peptide bonds. Thus, in a preferred example, the 5 peptide bonds of the homodetic pentacyclopeptide with sequence YSNSG (SEQ ID NO: 3) are trans. It has been shown that such a polypeptide is constrained into its β conformation at the amino acids YSNS (SEQ ID NO: 1) due to the trans peptide bonds. A particular cyclopeptide of the invention is that which has the three-dimensional conformation represented in FIG. 4, obtained by a random conformational investigation (see method, point A.5).

The choice of amino acid or amino acids composing the cyclopeptide, in addition to the sequence YSNS (SEQ ID NO: 1), must satisfy the structural and functional constraints indicated above. Among the structural constraints, the amino acid (or amino acids) is preferably of low charge or neutral and/or of low volume. Further, the choice of this amino acid (or these amino acids) must allow the formation of a β-bend at the amino acids YSNS (SEQ ID NO: 1) having a dihedral angle ψ+1 between −90° and −30° and a dihedral angle φ+2 between −120° and −60° (FIG. 1). Among the functional constraints, the resulting cyclopeptide must be capable of binding to αVβ3 integrin and/or of inhibiting the proliferation of melanoma cells and/or of inhibiting the migration of melanoma cells and/or of inhibiting angiogenesis. Thus, the amino acid residue glycine (G) may constitute an interesting choice because of the shortness of the side chain which may as a result increase the chances of cyclisation and reduce the risk of obtaining a peptide comprising peptide linkages in the cis position.

In a particular embodiment, the cyclopeptide of the invention is capable of being obtained by cyclisation of a linear peptide comprising the YSNS (SEQ ID NO: 1) sequence, particularly a linear peptide consisting of 4, 5, 6 or 7 amino acids, the cyclic sequence consisting of or comprising the peptide sequence Y-S-N-S (SEQ ID NO: 1). It is possible to obtain a cyclopeptide from the linear peptide with the same sequence using conventional cyclisatior methods. Briefly, the linear peptide is deprotected at its C-terminal then its N-terminal end. The cyclisation step is carried out in the liquid phase. After specific activation, the COOH function which has been rendered reactive may undergo nucleophilic attack of the basic nitrogen (N-terminal end) to result in the formation of the desired peptide bond. The cyclisation reaction is carried out at high dilution in order to eliminate any risk of dimerization. HPLC analysis or mass spectroscopy confirms the monomeric appearance of the peptide obtained (Thern B et al).

Preferably, the cyclopeptide is obtained by forming a peptide bond between the N- and C-terminal amino acids of the linear peptide.

More particularly, a cyclopentapeptide of the invention is capable of being obtained by forming a peptide bond between the amino acids Y and X or the linear peptide YSNSX (SEQ ID NO: 2).

Alternatively, the cyclopeptide may be obtained by forming a peptide bond between the terminal amino acids of the linear peptides SNSXY (SEQ ID NO: 7), NSXYS (SEQ ID ND: 8), SXYSN (SEQ ID NO: 9) and XYSNS (SEQ ID NO: 10) (in which X is as defined above)

In a particular embodiment, the formed peptide bond allows trans cyclisation, i.e. the Cα carbons (of the two amino acids bonded via said peptide bond) are positioned either side of the C—N bridge.

In one embodiment, all of the peptide bonds are trans, i.e. the peptide bonds between the amino acids of the linear peptide, but also the peptide bond allowing cyclisation.

In the particular case of a cyclopentapeptide with sequence YSNSG (SEQ ID NO: 3), cyclisation is obtained from the linear peptide YSNSG (SEQ ID NO: 3), by forming a peptide bond between the COOH carboxylic function of glycine (G) and the free amine NH₂ function of the N-terminal residue of tyrosine (Y).

In order to modify (to increase or to reduce) its stability or its bioavailability, the cyclopeptide of the invention may be modified before or after the cyclisation step. Thus, any of the amino acids of the cyclopeptide may undergo a chemical modification such as acetylation, alkylation, amidation, carboxylation, hydroxylation or methylation, or may have added thereto lipids (isoprenylation, palmitoylation, myristolylation or glypiation) or glucides (glycosylation).

Independently of or in combination with the chemical modifications, the cyclopeptide of the invention may also be subjected to covalent or non-covalent bonding with another molecule. Thus, in a particular embodiment, the cyclopeptide of the invention is coupled to a lipid group or bonded (possibly covalently) to a transporter or to a ligand. A cyclopeptide bonded to another molecule is defined in the context of the present invention as a hybrid compound.

However, for reasons primarily linked to bioavailability, the cyclopeptide of the invention is used in the form of a monomer.

A composition comprising at least one cyclopeptide (or a hybrid compound) described above also forms part of the invention. Thus, a composition of the invention comprises a single unique cyclopeptide, i.e. the composition comprises only one class of cyclopeptides with identical size and sequence and with peptide bonds which are all of the same type, for example all trans peptide bonds.

A particular composition in accordance with the invention comprises a cyclopentapeptide with sequence YSNSG (SEQ ID NO: 3) wherein all the peptide bonds are trans.

Alternatively, a composition of the invention comprises cyclopeptides wherein the size and/or sequence and/or the nature of the peptide bonds is different, provided that all of the cyclopeptides comprise the YSNS (SEQ ID NO: 1) sequence. As an example, a composition of the invention may comprise cyclopeptides with the same sequence but for which the nature: of the peptide bonds is different from one cyclopeptide to another, i.e. either cis or trans. The scope of the invention also encompasses a composition which comprises cyclopeptides of the invention of different size, for example a mixture of tetra, penta and/or hexa cyclopeptides, or with a different sequence, for example a mixture of cyclopentapeptides wherein the nature of the amino acid X is different from one cyclopeptide to another (for example a mixture of the cyclopentapeptides YSNSG (SEQ ID NO: 3) and YSNSA (SEQ ID NO: 4)).

The composition may also comprise any molecule which is capable of improving the biological activity of the cyclopeptide of the invention. The term “improve” encompasses both an increase in the biological activity of the cyclopeptide as defined above in in vitro or in vivo tests and a better biological activity at the target cells compared with a composition which comprises only the cyclopeptide, or increased bioavailability or stability. Thus, a molecule capable of encouraging transport of the cyclopeptide to its target cells or capable of reducing or retarding (over time) degradation of the cyclopeptide may be included in a composition of the invention. Similarly, a molecule which is capable of extending the biological activity of the cyclopeptide may be included in a composition of the invention; alternatively, the cyclopeptide of the invention is inserted in nanocapsules, which may be biodegradable, which thus improve bioavailability, protection and/or delivery of the cyclopeptide.

The invention also encompasses a composition as described in the preceding paragraphs further comprising at least one molecule of another type, which is biologically active in the treatment of cancer. The term “molecule which is biologically active in the treatment of cancer” means any molecule which may be involved in the treatment of cancer, in accordance with the definition given below. Examples of molecules which are biologically active in the treatment of cancer which may be cited are chemotherapeutic agents and immunotherapeutic agents which are conventionally used in the treatment of melanoma. The following may be cited in particular: dacarbazine, cisplatine, vindesine, fotemustine or nitrosoureas. Tumoral epitopes which are known to be specifically associated with tumour cells may also form part of the composition of the invention, in particular those associated with melanoma cells such as the Mage 1, 2 and 3, Bage, Gage 1 and 2 antigens or melanocytary differentiation antigens such as tyrosinase or Melan-A/MART-1. Finally, the composition may also include interleukin 2 or α interferon.

Finally, when one of the compositions described in the present application is administered systemically or locally, particularly by injection, the composition also comprises a pharmaceutically acceptable excipient, or a transporter and/or a vehicle.

The present application also concerns a method comprising in vivo administration of a cyclopeptide as defined above or a composition comprising it for the treatment of various types of cancer, more particularly tumour cells expressing the αVβ3 integrin molecule. Thus, a cyclopeptide or a composition comprising it may be used in the treatment of melanoma or bronchial cancer, breast cancer or prostate cancer. Thus, a cyclopeptide of the invention or a composition comprising it for use as a drug, and more particularly for use in the treatment of cancer such as the treatment of melanoma, also falls within the scope of the invention.

The term “cancer treatment” means the direct treatment of tumours, for example by reducing or stabilizing their number or their size (curative effect), but also by preventing the in situ progression of tumour cells or their diffusion, or the establishment of tumours; this also includes the treatment of deleterious effects linked to the presence of such tumours, in particular the attenuation of symptoms observed in a patient or an improvement in quality of life.

When different types of biologically active molecules form part of the composition with the cyclopeptides of the invention, a synergistic effect may be obtained against the tumour, in particular tumour progression, i.e. the combination of one or more biologically active molecules with one or more cyclopeptide(s) in accordance with the invention has an effect on tumour progression which is greater than the sum of the effects of the molecules and the cyclopeptides used separately. The biologically active molecules and the cyclopeptides of the invention may be administered together or separately over time.

The present application also concerns the in vitro or in vivo use of a cyclopeptide or a composition of the invention in reducing the proteolytic cascade associated with proMMP-2 or the plasminogen activation system (u-PA). The invention also encompasses a cyclopeptide or a composition of the invention for use in reducing the proteolytic cascade associated with proMMP-2 or the plasminogen activation system (u-PA).

Finally, in a further aspect of the invention, the cyclopeptide or the composition of the invention may also be used as an anti-angiogenic substance, both in vivo and in vitro. The term “anti-angiogenic substance” means a substance which has an inhibiting effect or reducing effect on the formation of blood vessels, preferably within tumours.

The anti-angiogenic properties of a cyclopeptide of the invention may, for example, be tested as described in the examples, on HUVEC cells. In particular, the effect of the cyclopeptide on cell migration may be investigated, for example by detecting the secretion of plasminogen activators in the presence of a cyclopeptide of the invention, or by assaying certain receptors of these activators, or by observing the effect of the cyclopeptide on the cell matrix.

The present application also concerns the use of a cyclopeptide or a composition of the invention in the manufacture of a drug for the treatment of cancer, and more particularly to treat various stages of melanomas. Thus, the cyclopeptide of the invention may be used to treat primary tumours, possibly before surgical excision, or to treat the surrounding regions to limit the first stage of invasion by local cutaneous metastases.

The cyclopeptide of the invention or a composition comprising it is administered to a patient by a subcutaneous route (s.c.), interdermal (i.d.), intramuscular (i.m.) or by intravenous injection (i.v.) or by oral administration. Because of its β-bend structure, the cyclopeptide is protected from proteases which provide it with great stability and bioavailability in vivo. As a result, the cyclopeptide of the invention is particularly suitable for oral administration of a composition containing it.

### DESCRIPTION OF THE DRAWINGS

FIG. 1: Representation of a β-bend formed by the amino acids YSNS (SEQ ID NO: 1) and the various characteristic dihedral angles of a β-bend, namely φ+1, ψ+1 φ+2 and ψ+2;

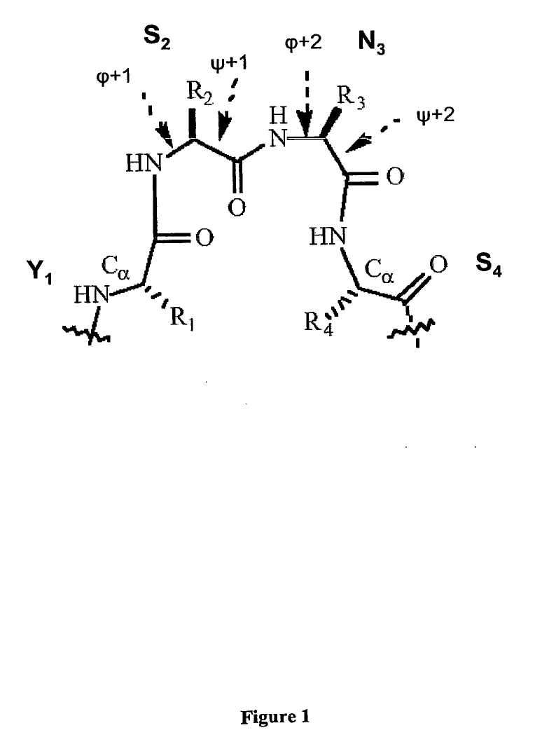

FIG. 2: Representation of 10 groups extracted from molecular dynamic simulation of the linear peptide YSNSG (SEQ ID NO: 3). Four structure classes can be distinguished: a β-bend structure on the amino acids YSNS (SEQ ID NO: 1) (groups 1, 3 and 5), a β-bend structure on the amino acids SNSG (group 2), intermediate structures (groups 4 and 6), and extended structures (groups 7, 8, 9 and 10). The amino acids are numbered from tyrosine (Y₁) to glycine (G₅).

FIG. 3: Nuclear magnetic resonance of the cyclopeptide YSNSG (SEQ ID NO: 3). Portions of the ROESY spectrum of the cyclopeptide YSNSG (SEQ ID NO: 3). Diagrams A and B describe the NH—NH and NH—Hα regions respectively. The abscissa and ordinate show the chemical displacements (in ppm: parts per million). The position of the spots corresponds to the correlations between the protons; thus, protons which are at a maximum of 4 Å produce a correlation on the 2D NMR spectrum;

FIG. 4: Minimum in vacuo conformational energy of the cyclopeptide YSNSG (SEQ ID NO: 3). This conformation was obtained from an in vacuo random conformational investigation using a distance-dependant dielectric constant and produced using NMR data; the nOes (nuclear Overhauser effect) observed experimentally which are included as the constraint in producing this conformation are shown as dotted lines. This conformation shares the minimum energy potential;

FIG. 5: Stable structure of cyclopeptide YSNSG (SEQ ID NO: 3) as observed during a 20 ns molecular dynamics trajectory. The simulation was carried out in explicit water and showed a stable β-bend structure. Panel A shows that the flexibility is reduced at the single side chain of tyrosine (Y₁). Panel B shows that the peptide structure has the particular feature of having a clear separation of the NH and C═O groups. Finally, panel C shows that the particular feature of the structure prevents the formation of internal hydrogen bonds;

FIG. 6: Mean proton-proton distances, extracted from the molecular dynamics simulation of the cyclopeptide YSNSG (SEQ ID NO: 3). The clear bars show the experimentally observed nOes; the black bars show the other nOes. This Figure shows a good correlation between the theoretical values and the experimentally observed values;

FIG. 7: Electrostatic potential of the cyclopeptide YSNSG (SEQ ID NO: 3). The isosurfaces at +7.5 kT/e (upper surface) and at −7.5 kT/e (lower surface) of the cyclopeptide YSNSG (SEQ ID NO: 3) (with T=300 K) were calculated and shown using VMD. This Figure shows the highly polarized aspect of the cyclopeptide;

FIG. 8: Circular dichroism of the cyclopeptide YSNSG (SEQ ID NO: 3). The various spectra were recorded at temperatures of 0° C., 20° C., 37° C. and 50° C. and are the result of 3 accumulations of a signal in a range of 190-250 nm;

FIG. 9: In vitro proliferation test for human melanoma cells UACC-903 in the presence of the cyclopeptide YSNSG (SEQ ID NO: 3). The UACC-903 cells were incubated for 48 hours in 24-well plates, with a control medium (negative control, white bar), a linear heptapeptide CNYYSNS (SEQ ID NO: 5) (positive control, black bar) or concentrations of cyclopeptide YSNSG (SEQ ID NO: 3) varying from 5 μM to 20 μm (grey bars). Cell proliferation was measured with the reagent Wst-1. *: significantly different from control (p<0.05); **: significantly different from control (p<0.01);

FIG. 10: In vitro migration test for UACC-903 human melanoma cells in the presence of the cyclopeptide YSNSG (SEQ ID NO: 3). The UACC-903 cells were incubated with a control medium (control, negative, white bar), a linear heptapeptide CNYYSNS (SEQ ID NO: 5) (positive control, black bar) or with the cyclopeptide YSNSG (SEQ ID NO: 3) at 20 μM (grey bar). After an incubation period of 48 hours, the migrated cells were stained with crystal violet and counted under reverse microscopy. The results were expressed as the mean of two independent experiments, each carried out in triplicate. **: significantly different from control (p<0.01);

FIG. 11: Effect of YSNSG (SEQ ID NO: 3) cyclopeptide on secretion of MMP-2 and MMP-9. The secretion of MMP-2 and MMP-9 was analyzed by zymography in the presence of gelatine (A), on a medium conditioned by UACC-903 cells in the absence of peptide (negative control, white bar), on cells treated with linear heptapeptide CNYYSNS (SEQ ID NO: 5) (20 μM) (positive control, black bar) or cells treated with concentrations of 5 μM to 20 μM of cyclopeptide (grey bars) YSNSG (SEQ ID NO: 3). The conditioned medium was collected and analyzed as described in the method. Quantification was carried out using Bio 1D software. B: quantification of proMMP2; C: quantification of proMMP-9 (A.U.: arbitrary unit);

FIG. 12: Effect of YSNSG (SEQ ID NO: 3) cyclopeptide on expression and activation of MMP-14. Expression of proMMP-14 (white bar) and MMP-14 (black bar) was studied by Western Blot (A) in a medium conditioned by UACC-903 cells in the absence of peptide (control), on cells treated with the linear heptapeptide CNYYSNS (SEQ ID NO: 5) (20 μm) or treated with the cyclopeptide YSNSG (SEQ ID NO: 3) at a concentration of 20 μM (cyclopeptide YSNSG (SEQ ID NO: 3)). The conditioned medium was recovered and analyzed as described in the methods. Quantification was carried out using Bio 1D software. (B);

FIG. 13: Effect of YSNSG (SEQ ID NO: 3) cyclopeptide on secretion of TIMP. The secretion of 3 tissue inhibitors of metalloproteinases (TIMP) was analyzed by reverse zymography (A) in a medium conditioned by control UACC-903 cells in the absence of peptide (negative control, white bar), on cells treated with the linear heptapeptide CNYYSNS (SEQ ID NO: 5) (20 μM) (positive control, black bar) or treated with cyclopeptide YSNSG (SEQ ID NO: 3) at a concentration from 5 μM to 20 μM (grey bars). The conditioned medium was recovered and analyzed as described in the methods. The quantification of TIMP-2 was carried out using Bio 1D software (B). **: significantly different from the control (p<0.01);

FIG. 14: Effect of YSNSG (SEQ ID NO: 3) cyclopeptide on the secretion of plasminogen activators. The secretion of u-PA (urokinase type plasminogen activator) and t-PA (tissue plasminogen activator) was analyzed by zymography in the presence of gelatine and plasminogen (A) in a medium conditioned by control UACC-903 cells in the absence of peptide (negative control, white bar), on cells treated with linear heptapeptide CNYYSNS (SEQ ID NO: 5) (20 μM) (positive control, black bar) or treated with the cyclopeptide YSNSG (SEQ ID NO: 3) at a concentration varying from 5 μM to 20 μM (grey bars). The conditioned medium was recovered and analyzed as described in the methods. The quantification of t-PA (B) and u-PA (C) was carried out using Bio 1D software. **: significantly different from the control (p<0.01);

FIG. 15: Effect of YSNSG (SEQ ID NO: 3) cyclopeptide on secretion of PAI-1 (plasminogen activator inhibitor-1). The secretion of PAI-1 was analyzed by Western Blot (A) in a medium conditioned by UACC-903 cells control in the absence of peptide (negative control, white bar), in cells treated with linear heptapeptide CNYYSNS (SEQ ID NO: 5) (20 μM) (positive control, black bar) or treated with YSNSG (SEQ ID NO: 3) cyclopeptide at a concentration from 5 μM to 20 μM (grey bars). The conditioned medium was recovered, concentrated and analyzed as described in the methods. PAI-1 (B) was quantified using Bio 1D software. **: significantly different from control (p<0.01);

FIG. 16: In vivo tumour growth in the presence of YSNSG (SEQ ID NO: 3) cyclopeptide. B16F1 cells were incubated for 15 minutes with a control medium (negative control, white diamonds), with the linear heptapeptide CNYYSNS (SEQ ID NO: 5) (20 μM) (positive control, black diamonds) or with the cyclopeptide YSNSG (SEQ ID NO: 3) at a concentration of 20 μM (grey diamonds), then injected subcutaneously into syngenic C57BL6 mice (2.5×10⁵ cells per mouse). The tumour volume was measured on the 20^{th} day;

FIG. 17: In vitro anti-angiogenic activity of cyclopeptide YSNSG (SEQ ID NO: 3). A: microscope photographs of the network of capillary pseudotubes of HMEC-1 endothelial cells after 24 hours incubation in the absence of peptide (control; 1), with the cyclopeptide YSNSG (SEQ ID NO: 3) at a concentration of 10 or 20 μM (2 and 3 respectively) or with the linear heptapeptide CNYYSNS (SEQ ID NO: 5) (20 μM) (positive control; 4). B: quantification of number of pseudotubes in photographs in A;

FIG. 18: In vitro inhibition of HUVEC cell migration in the presence of YSNSG (SEQ ID NO: 3) cyclopeptide. Microscope photographs of artificial wounds produced on a monolayer of endothelial cells at T0 and T48h in the absence of peptide (control) or in the presence of 20 μM of YSNSG (SEQ ID NO: 3) cyclopeptide;

FIG. 19: Effect of YSNSG (SEQ ID NO: 3) cyclopeptide on expression and activation of MMP-14 by HUVEC cells. The expression of proMMP-14 and MMP-14 was studied by Western blot (A). The HUVEC cells were cultivated in the absence (control) or in the presence of 20 μM of YSNSG (SEQ ID NO: 3) cyclopeptide and the corresponding membrane extracts were analyzed. Quantification was carried out using Bio 1D software (B);

FIG. 20: Effect of YSNSG (SEQ ID NO: 3) cyclopeptide on the secretion of plasminogen activators. The secretion of u-PA and t-PA was analyzed by zymography in the presence of gelatine and plasminogen (A) in a medium conditioned by HUVEC cells in the absence (control) or in the presence of 20 μM of cyclopeptide YSNSG (SEQ ID NO: 3). Quantification was carried out using Bio 1D software (B);

FIG. 21: Effect of YSNSG (SEQ ID NO: 3) cyclopeptide on the expression of the u-PA receptor: u-PAR by HUVEC cells. The secretion of u-PAR was analyzed by Western blot (A) in a medium conditioned by HUVEC cells in the absence (control) or in the presence of 20 μM of the cyclopeptide YSNSG (SEQ ID NO: 3). Quantification was carried out using Bio 1D software (B);

FIG. 22: Effect of YSNSG (SEQ ID NO: 3) cyclopeptide on the organization of the cytoskeleton of HUVEC cells. The cells were cultivated on glass slides in the absence (control) or in the presence of 20 μM of YSNSG (SEQ ID NO: 3) cyclopeptide. After 48 h of incubation, the cells were fixed, permeabilized, incubated with phalloidin (A) or with an anti-Phospho-FAK antibody (B) then with Hoechst-33342. The slides were observed under a confocal microscope;

FIG. 23: Effect of YSNSG (SEQ ID NO: 3) cyclopeptide on the distribution of β₁ integrin subunits on the surface of HUVEC cells. The cells were cultivated on glass slides in the absence (control) or in the presence of 20 μM of YSNSG (SEQ ID NO: 3) cyclopeptide. After 48 h of incubation, the cells were fixed and incubated in the presence of antibodies directed against the β₁ integrin subunit. The slides were observed using a confocal microscope.

### EXAMPLES

### A. Methods

### A.1. Reagents

All of the cell culture and molecular biology reagents were from Invitrogen (Cergy Pointoise, France); bovine serum albumin (BSA), gelatine and Matrigel (ECM gel) were from Sigma (Saint Quentin Fallavier, France). Human plasminogen derived from Calbiochem (VWR Int, Strasbourg, France); human anti-MMP-14 antibody was from Santa-Cruz Biotech (Tébubio, Le Perray en Yvelines, France). Human anti-PAI-1 antibody was from American Diagnostica (Neuville sur Oise, France). The reagents used in the experiments described in the present application are shown in Table 1.

### A.2. Peptides

The linear peptide NC1 [α3(IV) 185-191] with sequence CNYYSNS (SEQ ID NO: 5) was obtained by solid phase synthesis using a procedure derived from the FMOC synthesis; it was then purified by reverse phase HPLC with a C18 column and eluting on a gradient of acetonitrile in trifluoroacetic acid then freeze dried (Floquet et al). The cyclopeptide YSNSG (SEQ ID NO: 3) was ordered from Ansynth Service B.V. (Roosendaal, Netherlands). The cyclopeptide YSNSG (SEQ ID NO: 3) was in the acetate form and its purity was more than 95%, determined by reverse phase HPLC.

### A.3. Nuclear Magnetic Resonance (NMR)

The NMR spectra were recorded using a Bruker DRX 500 spectrometer. The samples were diluted in a mixture of D₂O:H₂O in a ratio of 9:1. TSP-d₄ (trimethylsilyl propanoic acid, sodium salt) was used as the internal reference for the chemical displacement. The water signal had been replaced by the WATERGATE sequence (Piotto M et al). The dependence of the chemical displacement of the amide protons was studied at 294K. Identification of the spin systems was carried out using TOCSY spectral analysis (mixing time 200 ms; mlevdpst19 pulse sequence). The distances between the protons were classified from the NOESY spectrum (mixing time 350 ms, noesyqpst19 pulse sequence). The acquisition matrix of the latter contained 512×2K points extended to 1K×4K by adding zeros. The cos² apodisation function was applied in two dimensions before 2D Fourier transformation.

TABLE 1
Supplier and reference of used reagents.

| | Supplier | Reference |
|---|---|---|
| Cell culture products | | |
| RPMI 1640 medium | Invitrogen, Cergy-Pontoise, France | 61870-010 |
| DMEM medium 4.5 g/l glucose | | 31966-021 |
| Trypsine/EDTA | | 15400-054 |
| Phosphate buffered saline | | 10010015 |
| Foetal calf serum | | 10270106 |
| Culture medium for ECGM | Promocell, Heidelberg, Germany | C-22020 |
| MV endothelial cells | | |
| Wst-1 reagent | Roche Diagnostics, Meylan, France | 11644807001 |
| Chemical products | | |
| Bovine serum albumin | Sigma, St Quentin Fallavier, France | A 9543 |
| Gelatine | | G8150 |
| Matrigel (ECM gel) | | E1270 |
| Transfer membrane | | P2938 |
| Immobilon - PVDF | | |
| Crystal violet | Aldrich, St Quentin Fallavier, France | 86,099-9 |
| Plasminogen EACA | Calbiochem, VWR, Strasbourg, France | 528178.50 |
| Pro-MMP-2 | Oncogene, VWR, Strasbourg, France | PF037 |
| TSP-d4 | Aldrich, St Quentin Fallavier, France | 18.033-5 |
| Antibody | | |
| Anti-MMP-14 | Chemicon, Euromedex, Souffelweyersheim, France | AB815 |
| Anti-PAI-1 | American Diagnostica, Neuville sur Oise, France | 395G |
| Cell culture medium | | |
| 24-well plates | Nunc, Dutscher, Brumath, France | 055429 |
| 25 cm² flasks | Nunc, Dutscher, Brumath, France | 055401 |
| Thincert 8 μM | Greiner, Dutscher, Brumath, France | 662638 |
| Laboratory animals | | |
| C57BL6 mice | Harlan, Gannat, France | |
| Synthetic peptides | | |
| YSNSG (SEQ ID NO: 3) cyclopeptide | Ansynth Service, Roosendaal, Netherlands | |
| Detection kits | | |
| ECL Chemiluminescence | Amersham, Saclay, France | RPN2132 |
| Biotrack ELISA TIMP-2 | | RPN2618 |
| Hyperfilm MP | | RPN3103K |

### A.4. Circular Dichroism (CD)

The circular dichroism spectra of the cyclopeptide YSNSG (SEQ ID NO: 3) were recorded using a JASCO J-810 CD spectropolarimeter provided with a Peltier PTC-623S temperature control system using a cell with an optical path length of 0.2 mm. The peptide was dissolved to a concentration of 0.2 mg/ml either in water (pH ˜3) or in a 20 mM potassium phosphate buffer so that the solution reached a pH of ˜7.3. The sweeps were acquired over a spectrum range of 190-250 nm at a temperature of 0° C., 20° C., 37° C. and 50° C., measuring a point every 0.1 nm.

### A.5. Random Conformational Investigation

Starting from a random conformation of the cyclopeptide YSNSG (SEQ ID NO: 3), conformational investigation of its structure was carried out in vacuo (isolation) with a distance-dependent dielectric constant using the CHARMM program and a force field (PARAM set 22) (Brooks et al; MacKerell et al) using NMR data; the experimentally observed interprotonic distances corresponding to the nuclear Overhauser effects (nOes) were constrained to an interval of 2.0 to 4.0 Å. Further, the amide protons of the peptide backbone which were not face to face were constrained to an interval of 4.0 to 8.0 Å. Since no Hα-Hα correlations were observed on the ROESY spectrum (data not shown), all of the peptide linkages were also constrained to their trans configuration (ω˜180°). Finally and in agreement with the Karplus relationship modified by Pardi et al, the angles φ of the two serine residues (S₂ and S₄) were constrained to about 120°±30° as a function of the ³J_{(NH—Hα)} coupling constants of 8.2 and 8.8 Hz respectively (the other residues shared values between 5.0 and 7.0 Hz).

At each step of the random conformational investigation protocol, all of the dihedral angles of the peptide backbone were distributed randomly; further, the corresponding structures in accordance with the above NMR analysis constraints were minimized. Each model generated was minimized using both the SD (steepest descent) algorithm and the ABNR (adopted based Newton Raphson) algorithm until an energy gradient of 10⁻⁵ kcal/mol was achieved. Three independent series of 1000 steps were carried out, by modifying the initial random seed of the random number generator. The 3000 minimized structures which were generated were grouped for analysis.

### A.6. Molecular Dynamics Simulation

The YSNSG (SEQ ID NO: 3) peptide (linear and cyclic) was also studied by molecular dynamics simulation. For the linear peptide, the starting point for the computations was a fully extended conformation. For the cyclopeptide, the simulation started from the conformation demanding a minimum of energy which had been obtained from the random conformational investigation. The peptides had initially been incorporated into water ensuring a 8 Å layer of water (TIP3P) on each side thereof, and the set of systems was minimized by 2000 steps of the conjugated gradient algorithm and brought to ambient temperature (300K) using the NAMD program (Phillips et al; Kale et al). The two simulations (linear or cyclic peptide) were carried out in an NPT assembly (1 atm, 300K) using the Verlet algorithm and a 10⁻¹⁵ S integration step. The electrostatic interactions were processed by applying a smoothing function to the conventional potential between 10 and 12 Å (threshold value). A conformational analysis was carried out over the whole of the simulation which was 20 ns, sampling the peptide structure every 10 ps. These models were grouped into a reduced number of groups using the NMRCLUST software (Kelley et al) and a threshold of 2.5 Å for the group threshold value.

### A.7. Animals

Female C57BL6 mice (mean body weight 18 to 20 g) were purchased from Harlan France (Gannat, France). The animals were placed in individual cages at a constant temperature and humidity; food and water were supplied ad libitum. All of the mice underwent a 1 week acclimatization period before commencing the experiments. The in vivo experiments were carried out in accordance with the recommendations of the Centre National de la recherché Scientifique (CNRS, National Centre for Scientific Research).

### A.8. Cell Culture and Culture Conditions

B16F1 cells, a metastasic sub-line of B16 murine melanoma pulmonary cells (d-Dr M Grégoire, INSERM U419, Nantes, France) were cultivated in RPMI 1640 medium, supplemented with 5% foetal calf serum (FCS) in 25 cm² flasks (Nunclon, VWR Int, Strasbourg, France) in a humid atmosphere containing 5% CO₂. UACC-903 cells, a melanoma cell line (Dr J Trent, Phoenix, Ariz.) were cultivated in DMEM medium containing 4.5 g/l glucose, supplemented with 5% FCS in 25 cm² flasks (Nunclon) in a moist atmosphere containing 5% CO₂. HMEC-1 cells (human microvascular entothelial cells; E W Ades, Center for Disease Control and Prevention, Atlanta, Ga.) and HUVEC cells (human umbilical vein endothelial cells) were cultivated in endothelial cell growth medium (ECGM) supplemented with ECGS/H at 0.4% (by weight), 2% (by volume) foetal calf serum, epidermal growth factor (EGF) 10 ng/ml, 1 μg/ml of hydrocortisone, 50 ng/ml of amphotericin B and 50 μg/ml of gentamicin.

### A.9. In Vitro Proliferation Test

Cell proliferation was determined using the reagent Wst-1 following the manufacturer's instructions. This reagent contains a tetrazolium salt which is reduced by mitochondrial oxido-reductases and allows the proliferation and viability of cells to be evaluated. Its reduction gives rise to a soluble formazan derivative which is yellow in colour. Before reaching confluence, the cells were washed twice with a saline solution buffered with phosphate (phosphate buffered saline; PBS) to remove SVF residues and incubated for 48 hours in DMEM medium, in the presence or absence of synthetic peptides at concentrations of 0 to 20 μM. The medium was recovered then centrifuged at 500 g for 10 min at 4° C. to remove cell debris. The protein contents of the medium obtained was determined using Bradford's method using SAB as the reference.

### A.10. In Vitro Invasion Test

Cell invasion was tested in modified Boyden chambers (ThinCert®, a cell culture insert for 24-well plates, internal diameter 8.36 mm, 8 μm pore, Greiner Bio-one, Courtaboeuf, France). Briefly, 5×10⁴ cells were suspended in DMEM medium without FCS and containing 0.2% BSA and seeded onto membranes spread with Matrigel (30 μg/cm²). DMEM medium supplemented with 10% FCS and 2% BSA were used as the chemo-attracting agent. After an incubation period of 48 hours, the cells were fixed with methanol and stained with crystal violet for 15 min. The cells remaining on the upper surface of the membranes were eliminated by scraping and those on the lower surface were counted using a reverse microscope (Pasco et al, 2000(a)). The counted cells corresponded to the cells which had crossed the Matrigel mat then the porous membrane of the Boyden chamber; they thus reflected the migration and invasion capacities of the tumour cells through an extracellular matrix or a basal membrane, reproduced in this case by the Matrigel.

### A.11. Zymographic Analysis

### Zymography in the Presence of Gelatine

The expression of MMP-2 and MMP-9 in a conditioned medium of UACC-903 cells, treated or not treated with synthetic peptides, was analyzed by zymography in the presence of gelatine as described above (Pasco et al, 2000(a)). The secretion of TIMP-2 was analyzed in a conditioned medium of UACC-903 cells by reverse zymography in the presence of gelatine, as described above (Pascal et al, 2000(a)).

### Zymography in the Presence of Gelatine and Plasminogen

To determine the plasminogen activities, conditioned media of UACC-903 cells were analyzed by polyacrylamide gel electrophoresis in the presence of SDS and containing 1 mg/ml of gelatine and 10 μg/ml of plasminogen. After electrophoresis, the gels were incubated overnight at ambient temperature in a 100 mM glycine, 5 mM EDTA, pH 8.0 buffer. The gelatinolytic activity resulting from plasminogen activation was demonstrated by lysis zones which were white after staining the gel with Coomassie gel (Pasco et al, 2004).

### A.12. Western Blot

The samples underwent 10% polyacrylamide gel electrophoresis in the presence of 0.1% SDS then were transferred onto Immobilon-P membranes (Millipore, St Quentin en Yvelines, France). The membranes were saturated with 5% freeze dried skimmed milk, Tween 20 0.1% in a 50 mM Tris-HCl, 150 mM NaCl buffer, pH 7.5 (TBS) for 2 hours at ambient temperature. The membranes were then incubated overnight at 4° C. with anti-MMP-14, anti-PAI-1 and anti-u-PAR antibodies then incubated for one hour at ambient temperature with a secondary antibody (anti-IgG) conjugated with peroxidase. The immune complexes were visualized with an ECL chemoluminescence detection kit.

### A.13. Measurement of In Vivo Tumour Growth

A suspension of B16F1 cells (2.5×10⁵ cells in RPMI 1640 medium) which may or may not have been pre-treated with the cyclopeptide YSNSG (SEQ ID NO: 3) (20 μM) was injected subcutaneously into the left flank of syngenic mice from a different series. The mice were sacrificed on day 20 and the tumour size was measured. The volume of the tumours was determined by the relationship V=½A×B² in which A and B respectively represent the largest and the smallest dimension of the tumour (Wald et al).

### A.14. Statistical Analyses

The statistical analyses were carried out using a Student t test. The results are expressed as the mean±1 standard deviation. For the in vivo experiments, the volumes of the primary tumours were analyzed statistically using The Mann and Whitney non-parametric u test and the parametric Student t test, compared with the mice selected for a corresponding weight.

### A.15. Measurement of Anti-Angiogenic Effect

The in vitro anti-angiogenic activity of the cyclopeptide YSNSG (SEQ ID NO: 3) was measured on the formation of capillary pseudotubes by endothelial HMEC-1 or HUVEC cells deposited on a Matrigel mat.

A solution of Matrigel (ECM gel: 10 mg/ml) was deposited in a sterile manner in 24-well plates (Nunclon) in an amount of 250 μl per well onto piled ice. Gel formation was obtained by incubating the plate at 37° C. for 30 min. HMEC-1 cells or HUVEC cells at sub-confluence were detached from their plastic culture support by adding trypsin/EDTA then taken up into suspension in the culture medium for endothelial cells and diluted to a concentration of 2×10⁵ cells per ml. 100 μl volumes of this cell suspension were deposited on the gel surface. After incubating for 1 hour at 37° C. to allow cell adhesion, the various peptides were added in solution to 400 μl of the culture medium (final concentration 10 or 20 μM). Pseudotube formation was observed under the microscope and photographed, after incubating for 24 hours. Quantification was carried out using image analysis software.

### A.16. Scar Wounding Technique

1×10⁵ endothelial cells were seeded into 24-well plates and incubated for 24 hours at 37° C. A wound was made in the cellular monolayer at the centre of the well using a 200 μl cone point. The cells were washed with PBS to eliminate cell debris then incubated in the presence or absence of the cyclopeptide YSNSG (SEQ ID NO: 3). Closing of the wound, a witness to the migratory properties of the cells, was observed under the microscope and photographed after 48 hours incubation.

### A.17. Hoechst-33342 Cytochemical Staining

The cells were cultivated in 24-well plates in the presence or absence of YSNSG (SEQ ID NO: 3) cyclopeptide. After 24 hours incubation, the cells were rinsed twice with a PBS solution. At this stage, Hoechst-33342 fluorochrome (50 μg/ml) diluted in PBS was added. After 15 minutes incubation at 37° C., 5% CO₂, the cell nuclei were observed using a fluorescence microscope (λ excitation=346 nm; λ emission=460 nm) then photographed.

### A.18. Labelling Actin F

The cells were seeded onto sterile glass slides placed in a 24-well plate (2×10⁴ cells/well) in a suitable medium containing 5% of serum, in the presence or absence of the cyclopeptide YSNSG (SEQ ID NO: 3). After 24 and 48 hours incubation, the cells were fixed with 3.7% formaldehyde for 10 minutes at ambient temperature and permeabilized for 3 minutes with an acetone solution at −20° C. Three 5 minute washes in PBS were carried out, then the cells were incubated for 30 minutes at ambient temperature in a saturation solution (PBS containing 1% bovine serum albumin). The cells were then incubated for 1 hour with phalloidin coupled with Alexa Fluor® 568 (diluted 1/40 in PBS containing 1% BSA) then washed 3 times for 5 minutes with PBS. The slides were observed with a confocal microscope and photographed.

### A.19. Phospho-FAK and β1 Integrin Immunolabelling

The cells were seeded onto sterile glass slides placed in 24-well plates (2×10⁴ cells/well) in a suitable medium containing 5% serum in the presence or absence of YSNSG (SEQ ID NO: 3) cyclopeptide. After 24 and 48 hours incubation, the cells were fixed for 15 minutes in methanol. Three washes of 5 minutes in TBS-T were carried out, then the cells were incubated for 30 minutes at ambient temperature in a saturation solution (TBS-T containing 3% BSA). The cells were then incubated for 2 hours at ambient temperature in the presence of primary antibody diluted to 1/100 in TBS-T containing 3% BSA. After 5×5 minute washes in TBS-T, the (cells were incubated with the corresponding secondary antibody coupled to Alexa Fluor® 488 diluted to 1/1000 in TBS-T containing 3% of bovine serum albumin. The cells were then washed for 3×5 minutes with TBS-T. The slides were observed with a confocal microscope and photographed.

### B. Results

### B.1. Structure of Linear Peptide YSNSG (SEQ ID NO: 3)

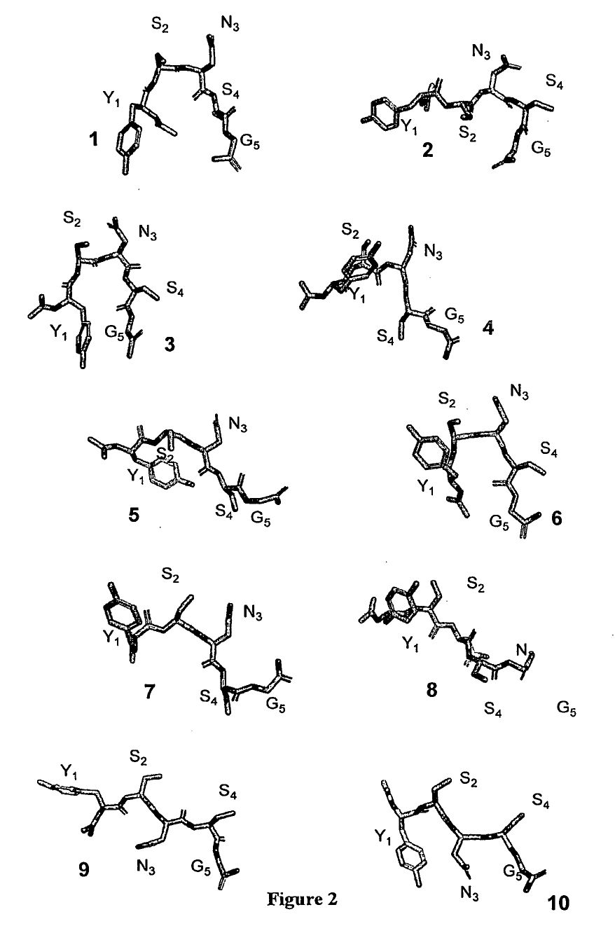

As the starting point of the linear peptide study, a molecular dynamic simulation was carried out at 300K on the linear peptide YSNSG (SEQ ID NO: 3) with explicit representation of the solvent and starting from a fully extended conformation (φ=180°; ψ=180°). The simulation lasted 20 ns. This experiment showed up ten groups over the whole trajectory, representative of the various structures shown in FIG. 2. Note among these groups the presence of YSNS (SEQ ID NO: 1) β-bend (groups 1, 3 and 5), β-bend SNSG (group 2), intermediate structures (groups 4 and 6) and extended or quasi-extended structures (groups 7, 8, 9 and 10). Because of their parameters φ and ψ, the YSNS (SEQ ID NO: 1) and SNSG β-bends were very close to type I β-bends (central residues of bends located at the region of the α helix of the Ramachandran diagram). In view of these results and maintenance of the β-bend structure despite the addition of a glycine residue, the production of a YSNSG (SEQ ID NO: 3) cyclopeptide was thus envisaged.

### B.2. Structure of YSNSG (SEQ ID NO: 3) Cyclopeptide

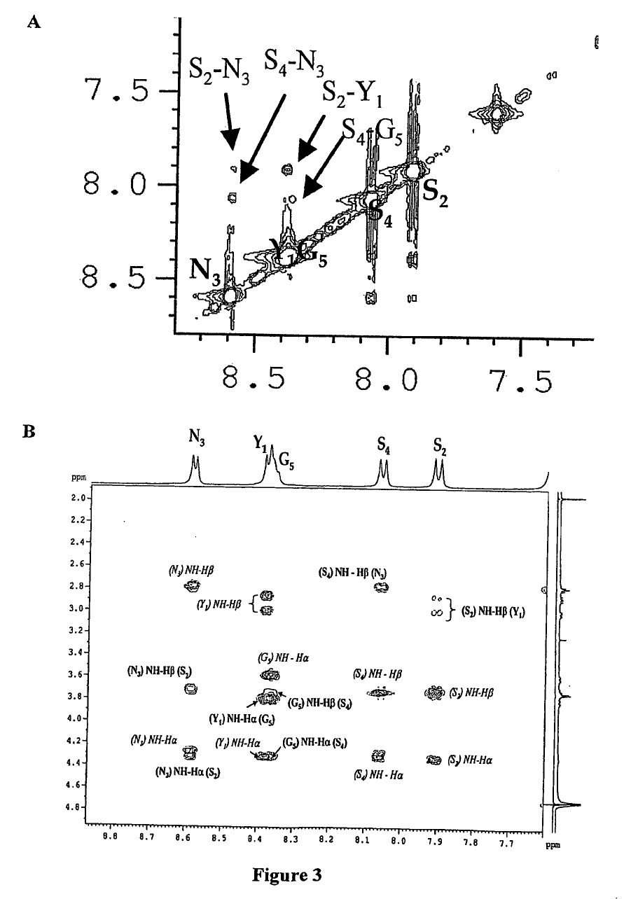

The structure of the cyclopeptide YSNSG (SEQ ID NO: 3) was studied firstly by nuclear magnetic resonance (NMR). After attributing all of the proton signals on the TOCSY spectrum, one of the most interesting pieces of information derived from the ROESY spectrum which clearly indicated a correlation between the amide protons of the backbone of the sequential residues. However, no correlation was observed with the non sequential residues (FIG. 3A). Several other nOes were also revealed by the various spectra in Nh-Hα regions (FIG. 3B).

A random conformational study of the structure of the peptide was carried out using the NMR data (see Apparatus and Methods). Interestingly, the three independent tests converged towards an identical conformation of very low energy with an RMSD for all atoms of less than 0.2 Å.

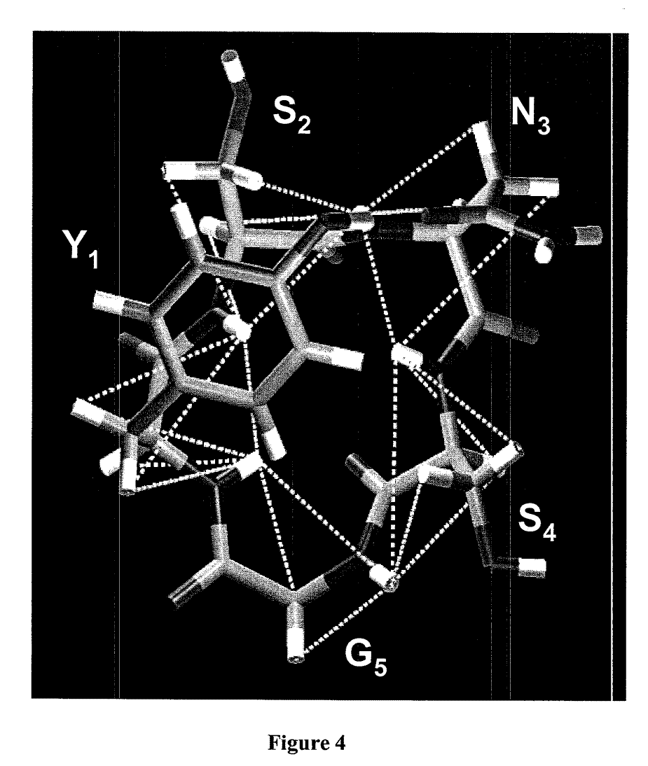

This structure (FIG. 4) shares a β-bend over the residues YSNS (SEQ ID NO: 1) with a Cα(Y₁)-Cα(S₄) distance of less than 7.0 Å. The in vacuo structure was stabilized by hydrogen bonds between the amide protons of Y₁ and the C═O structure of S₄ forming a γ bend on the S₄GY₁ residues and the hydrogen bonds between the hydroxyl group of Y₁ and the amide function of the side chain of N₃.

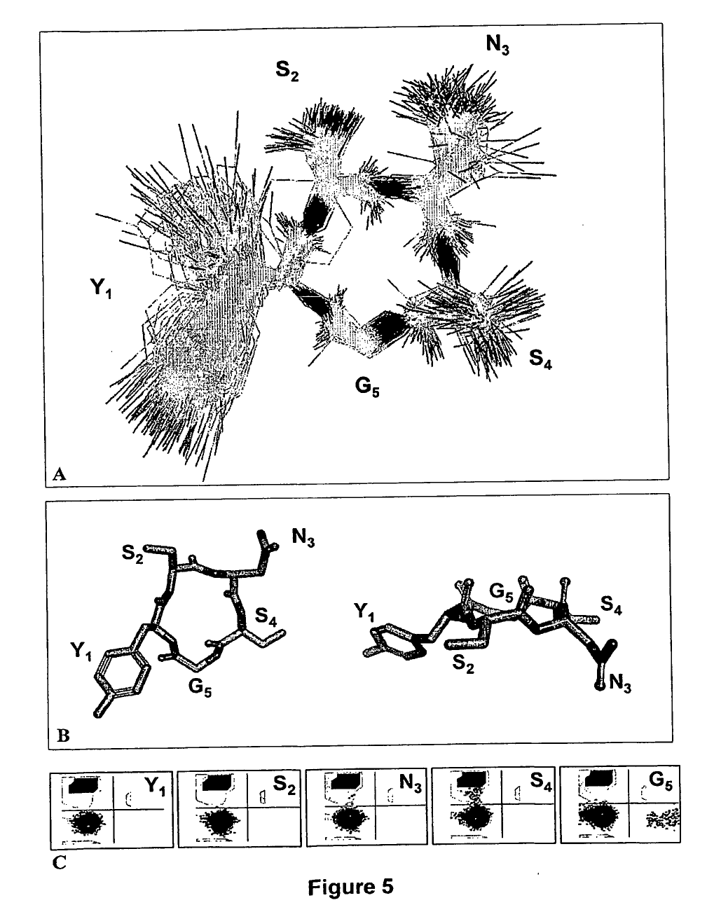

This conformation was then used as a starting point in an unconstrained molecular dynamics simulation which was carried out under the same conditions as that carried out for the linear peptide (explicit water, 300K). As expected, the cyclopeptide was observed in the form of a highly constrained structure as indicated by the RMSD value of less than 0.5 Å over the atoms of the backbone all along the given trajectory (not shown). The γ bend described above disappeared rapidly (during the heating phase) to reach a stable conformation in the first moments of simulation. The flexibility of the peptide was then reduced to explore the various rotamers of the tyrosine. The corresponding conformation was characterized by the YSNS (SEQ ID NO: 1) β-bend during the whole trajectory (FIG. 5). The diagram of the various angles of the backbone φ/ψ of the peptide along the whole trajectory also showed that these five residues were often localized in an α helix region of the Ramachandran diagram, making of the YSNS (SEQ ID NO: 1) tetrapeptide a structure close to a type I β-bend. However, this bend does not belong to any canonical type β-bend with a mean φ/ψ of (−90° C.; −66° C.) for both residues S₂ and N₃.

This conformation is very close to that of group 5 described for the linear peptide with a mean RMSD of approximately 1.5 Å calculated for the atoms of the backbone.

B.3. Comparison with Experimental Data

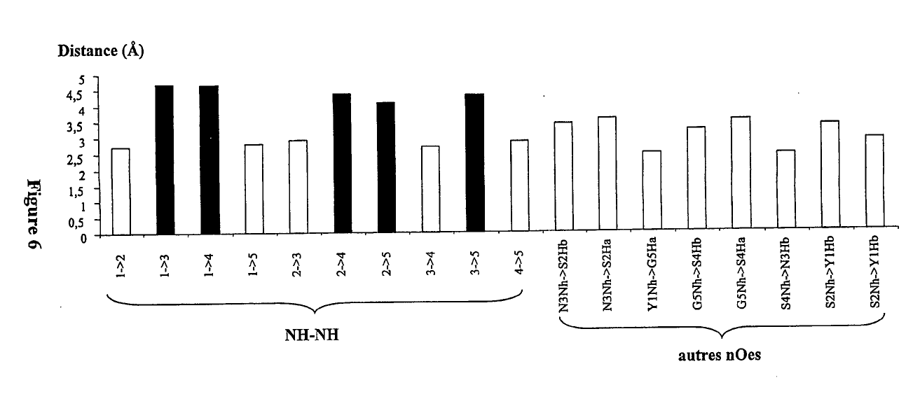

In order to compare the experimental and theoretical results in more detail, a diagram of the mean proton/proton distances was produced (shown in FIG. 6). The mean distances were extracted from the molecular dynamics simulations. Through this Figure, we can see that the experimentally observed nOes, particularly between the amide protons of the sequential residues, correspond to mean distances of less than 4 Å. In contrast, the non-facing amide protons of the backbone correspond to distances of more than 4 Å. Further, the mean observed value for the dihedral angles of the two serine residues was approximately −90° C., in agreement with the NMR experiment.

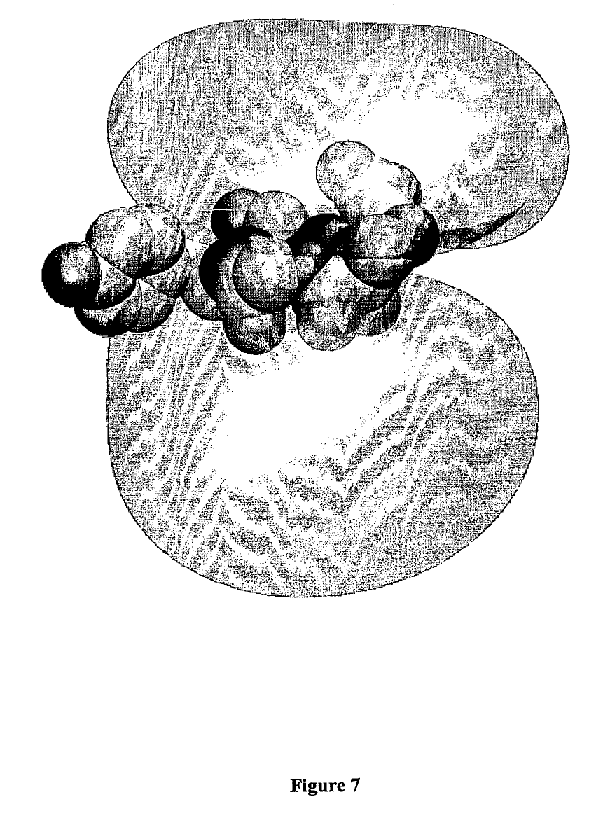

A particular aspect of the conformation of the peptide observed during the molecular dynamic trajectory was that all of the NH functions of the backbone were collected on the same face of the peptide while all of the carbonyl functions were collected on the other face (FIG. 7).

This particular conformation prevents the formation of an internal hydrogen bond in the backbone. This result was in agreement with the experimental data NMR data. In fact, the temperature coefficients of the amide proteins of the peptide which were recorded between 295K and 320K did not predict any internal hydrogen bonding, with values of −10.0, −4.6, −7.0, −4.6 and −6.1 ppb/K for Y₁, S₂, N₃, S₄ and G₅ respectively (values of less than −4.5 ppb/K are often associated with the absence of a hydrogen bond). Because of this feature, the peptide appears to be highly polarized, as can be seen in FIG. 6 which records the electrostatic potential of the peptide (isovalues of 7.5 and −7.5 kT/e in the upper position and lower position respectively). The charge distribution was calculated using the default parameters of the “Particle Mesh Ewald” method (Essmann et al) as carried out by VMD (Humphrey et al). Interestingly, the positive charges encompassing the side chains of the SNS residues are known to play a crucial role in the biological activity of the peptide and it derivatives, a feature which may also be important for fixing the peptide to αVβ3 integrin (Pasco et al; 2000(b).

### B.4. Characterisation of the Secondary Structure of YSNSG (SEQ ID NO: 3) Cyclopeptide by Circular Dichroism Spectroscopy

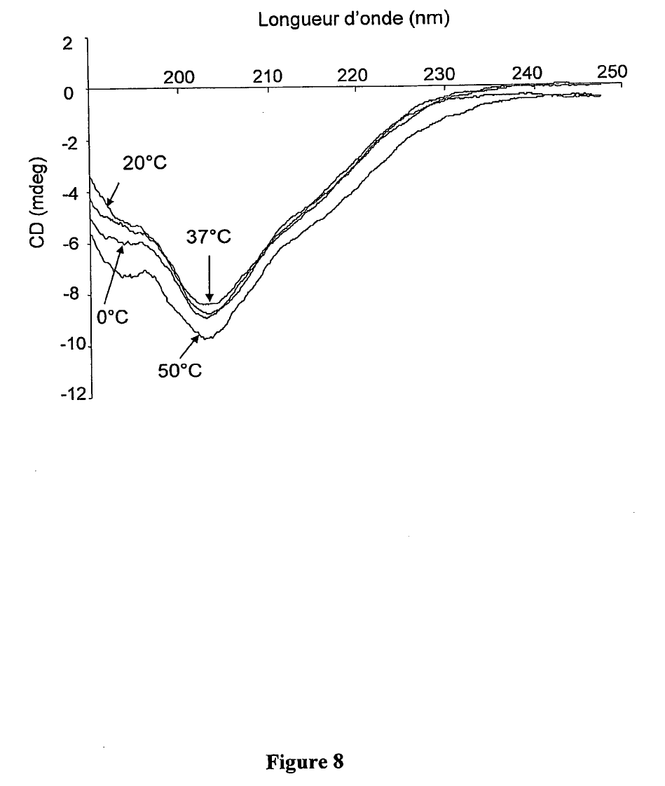

FIG. 8 shows the circular dichroism (CD) spectrum of the cyclopeptide YSNSG (SEQ ID NO: 3) recorded at a pH of 7 and at temperatures of 0° C., 20° C., 37° C. and 50° C. The signal obtained does not clearly show the presence of a well-structured conformation which is neither totally random nor α nor β. The same results were observed without adding buffer at a pH of approximately 3; this shows that the peptide is less sensitive to temperature and to pH, probably due to its highly constrained aspect.

The CD form observed is not characteristic of any conventional β-bend conformation (type I, type II or type VIII); this is probably due to the distorted φ/ψ parameters of the peptide compared with a type I β-bend.

Since the NMR, the molecular model and the CD results have shown that the cyclopeptide YSNSG (SEQ ID NO: 3) adopted the expected β-bend conformation, its biological activity was compared with that of the linear peptide CNYYSNS (SEQ ID NO: 5).

### B.5. Inhibition of In Vitro Proliferation of UACC-903 Human Melanoma Cells by the Cyclopeptide YSNSG (SEQ ID NO: 3)

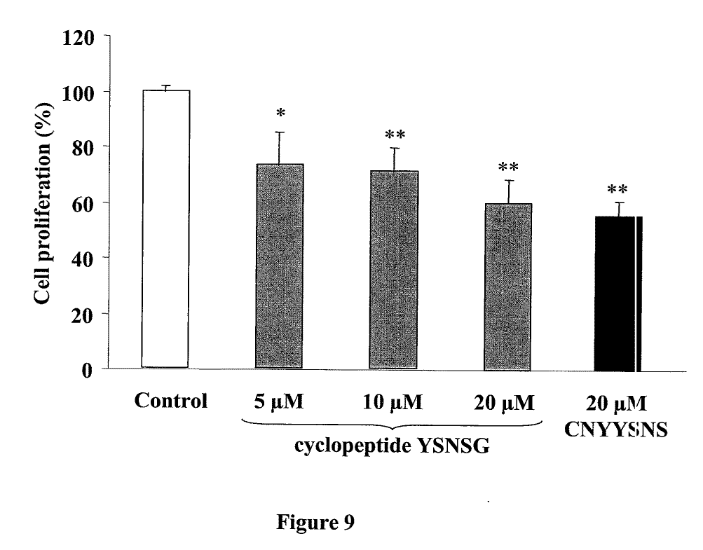

In vitro proliferation of UACC-903 human melanoma cells was significantly inhibited by the linear heptapeptide CNYYSNS (SEQ ID NO: 5) (−45%). The cyclopeptide YSNSG (SEQ ID NO: 3) at concentrations of 5, 10 and 20 μM also inhibited the proliferation of UACC-903 melanoma cells at 27%, 29% and 40% respectively (FIG. 9). The cyclopeptide is also approximately as active as the natural peptide at the same concentrations.

### B.6. Inhibition of In Vitro Migration of UACC-903 Melanoma Cells by the Cyclopeptide YSNSG (SEQ ID NO: 3)

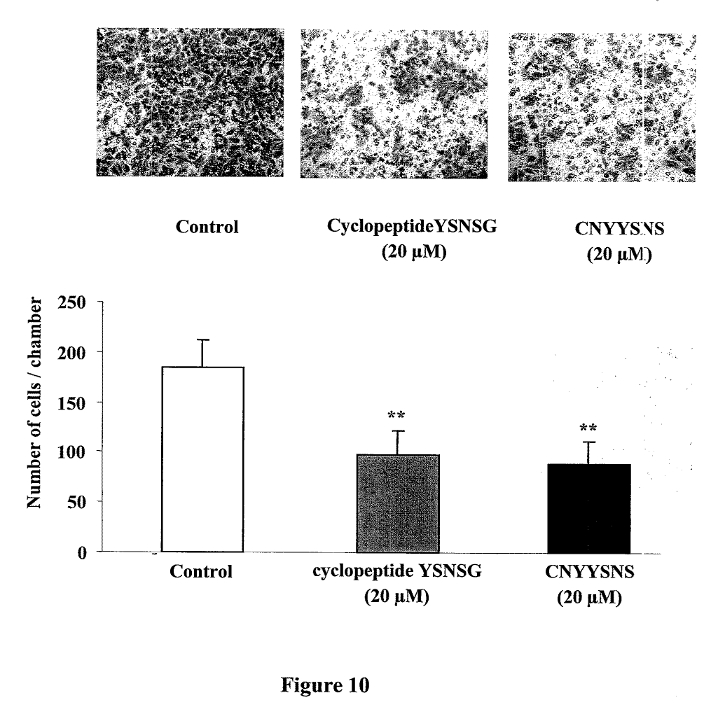

The effect of the cyclopeptide YSNSG (SEQ ID NO: 3) on the migration of UACC-903 cells on a basal membrane reconstituted in vitro was analyzed using Matrigel membranes and 10% FCS as a chemoattractive agent. Migration was measured after 48 hours. The YSNSG (SEQ ID NO: 3) cyclopeptide induced a substantial reduction in the migration of UACC-903 cells (−47%), similar to the linear heptapeptide CNYYSNS (SEQ ID NO: 5) at the same concentration (FIG. 10).

B.7. Effects of YSNSG (SEQ ID NO: 3) Cyclopeptide on Matrix Metalloproteinases and their Inhibitors

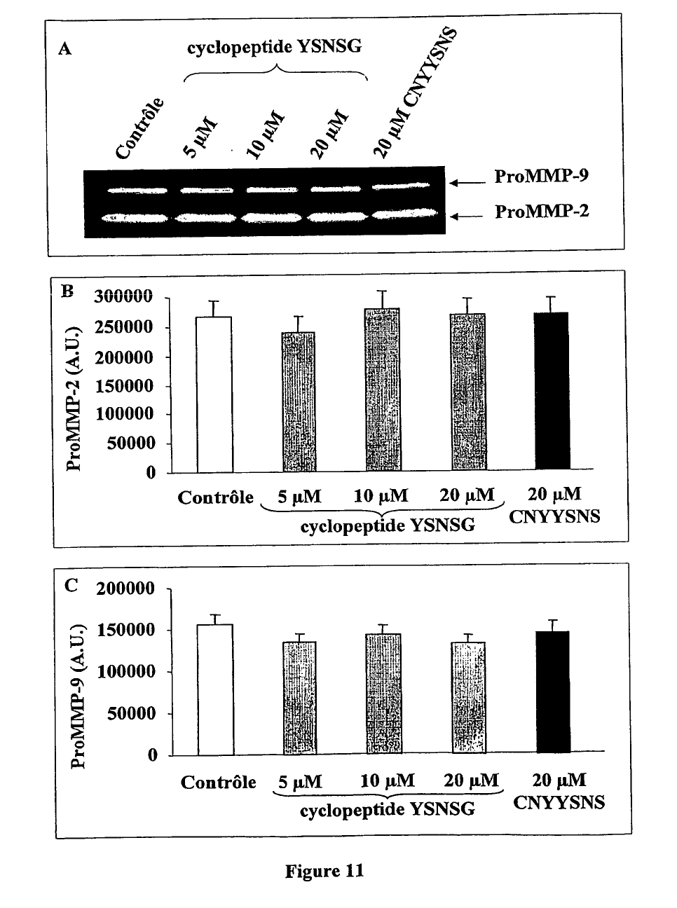

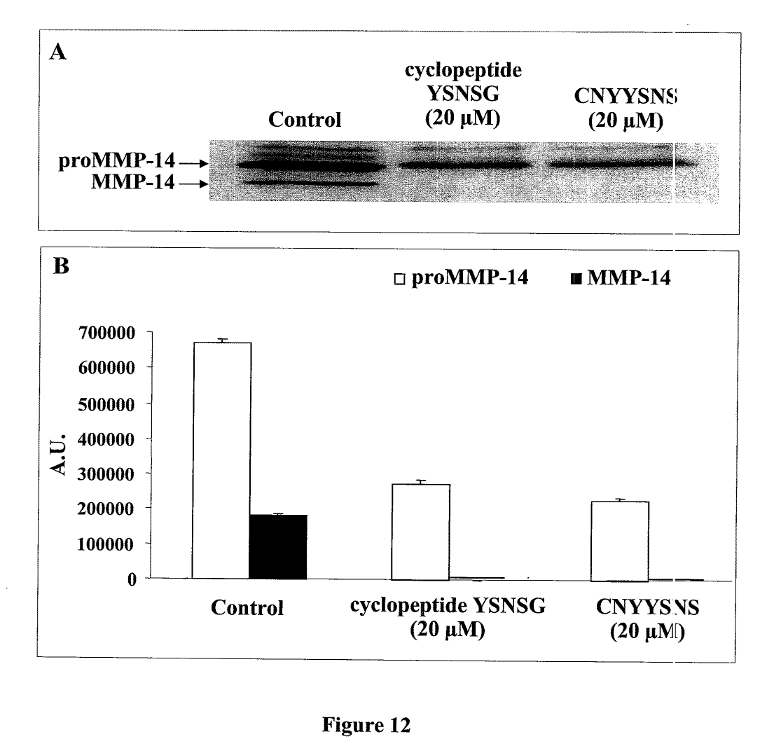

No significant change in the secretion of MMP-2 and MMP-9 was detected by zymography in the presence of gelatine after treatment of UACC-903 cells with the cyclopeptide YSNSG (SEQ ID NO: 3) or the linear peptide CNYYSNS (SEQ ID NO: 5) (FIG. 11). However, the cyclopeptide YSNSG (SEQ ID NO: 3) strongly inhibited the expression of proMMP-14, as can be seen by the Western blot analysis (FIG. 12). The activation of proMMP-14 was also completely abolished. The inhibiting effects of the cyclopeptide have the same intensity as that observed with the natural peptide.

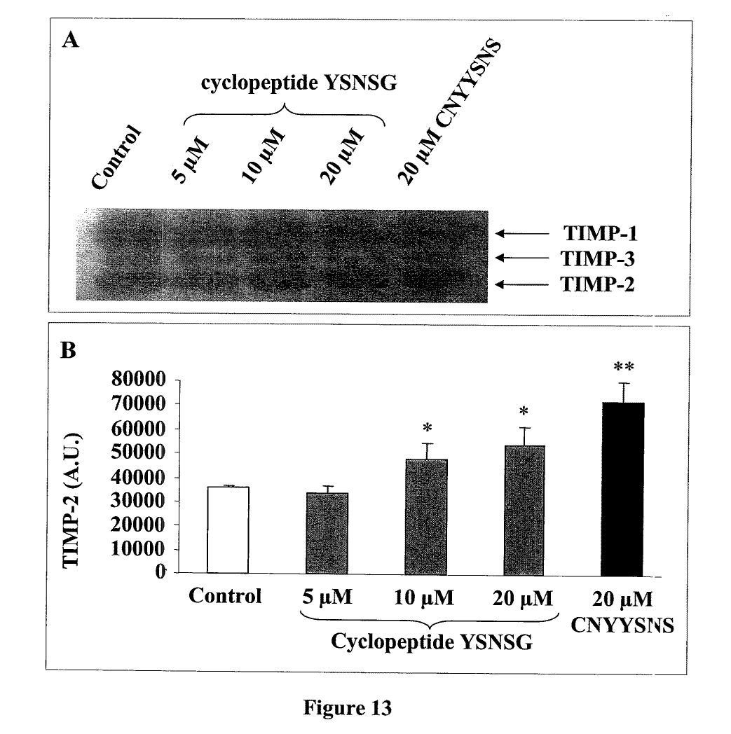

The secretion of TIMP in a conditioned culture medium was analyzed by reverse zymography (FIG. 13A). A dose-dependent increase in the secretion of TIMP-2 was observed in cultures incubated with the cyclopeptide YSNSG (SEQ ID NO: 3) and also with the linear peptide CNYYSNS (SEQ ID NO: 5) (FIG. 13B). This result was confirmed using the ELISA Biotrak system (Amersham Biosciences, Orsay, France) (data not shown). Secretion of TIMP-1 and TIMP-3 was not modified.

### B.8. Effects of YSNSG (SEQ ID NO: 3) Cyclopeptide on the Plasminogen Activation System

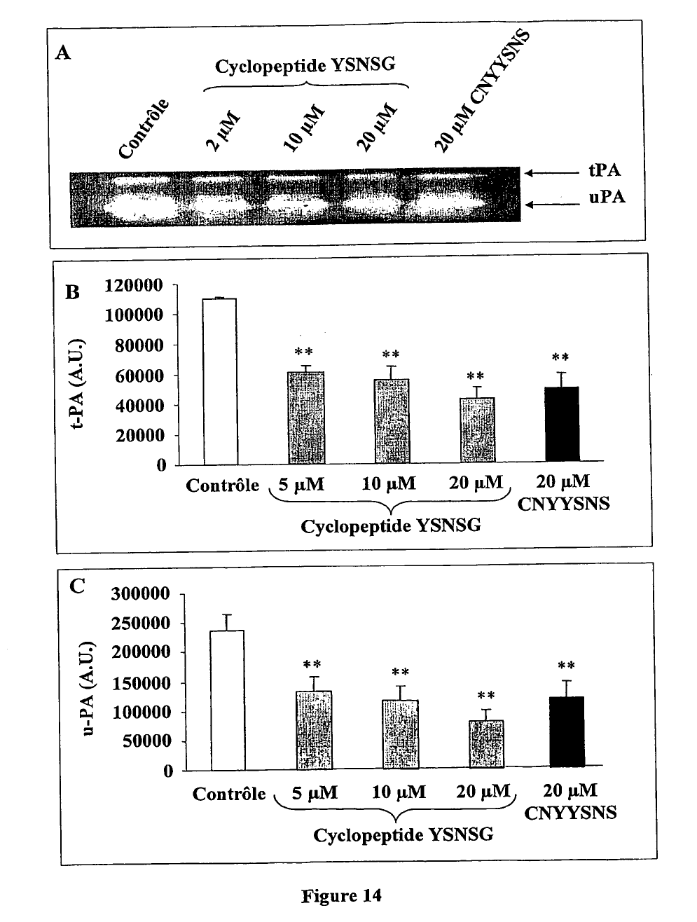

The secretion of u-PA and t-PA, the two principal activators of plasminogen, was analyzed by zymography in the presence of gelatine and plasminogen (FIG. 14). The treatment of UACC-903 cells with the cyclopeptide YSNSG (SEQ ID NO: 3) induced a dose-dependent reduction in the secretion of u-PA and t-PA which was greater than the reduction observed with the linear heptapeptide CNYYSNS (SEQ ID NO: 5).

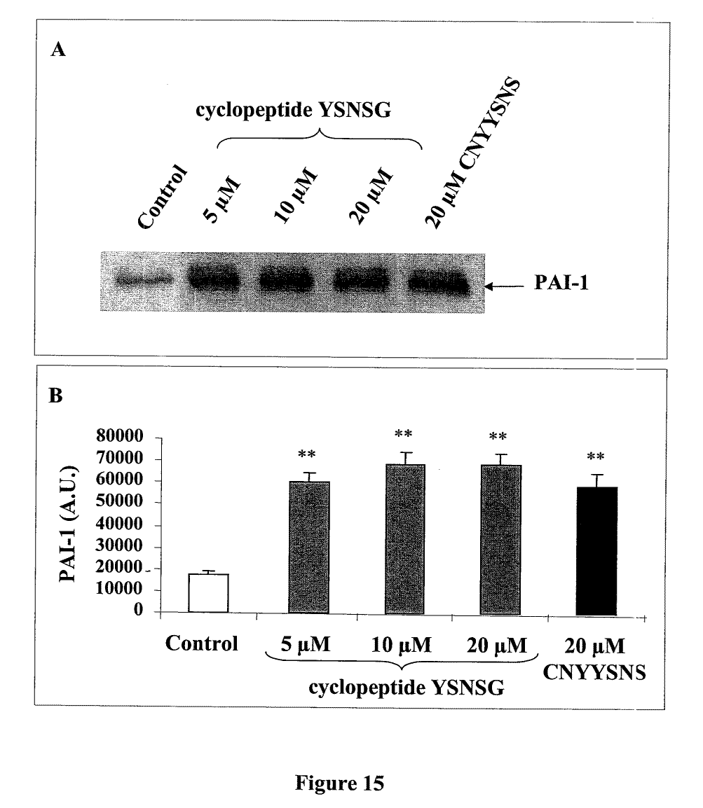

The secretion of PAI-1, an inhibitor of u-PA and t-PA, was analyzed by Western blot (FIG. 15). The treatment of UACC-903 cells with the cyclopeptide YSNSG (SEQ ID NO: 3) induced a large increase in the secretion of PAI-1, greater than that observed with the heptapeptide CNYYSNS (SEQ ID NO: 5).

### B.9. Inhibition of In Vivo Tumour Growth by the Cyclopeptide YSNSG (SEQ ID NO: 3)

The in vitro results indicate that the cyclopeptide YSNSG (SEQ ID NO: 3) inhibits the degradation of the extracellular matrix and at the same time inhibits the invasion of tumour cells in a manner which is as effective as with the natural linear peptide CNYYSNS (SEQ ID NO: 5). As a consequence, a study of its in vivo effects was carried out.

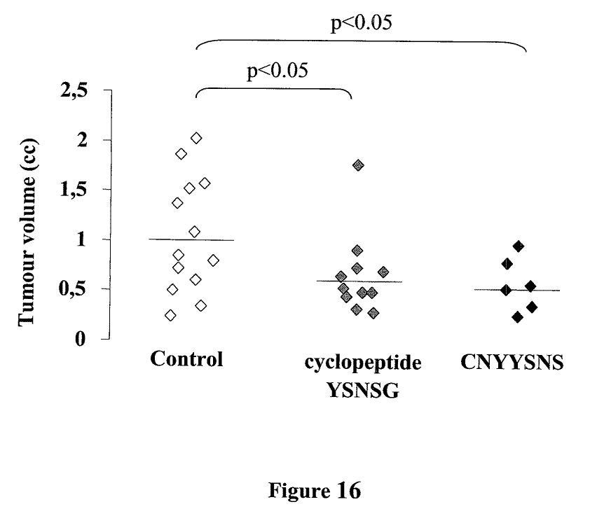

The effects of the cyclopeptide YSNSG (SEQ ID NO: 3) on in vivo tumour growth were studied in a murine melanoma model. B16F1 murine melanoma cells pre-incubated with the cyclopeptide YSNSG (SEQ ID NO: 3) or not were injected subcutaneously into the left flank of syngenic C57BL6 mice and the tumour volume was measured on day 20. The untreated B16F1 cells induced the development of subcutaneous tumours. Pre-incubation of tumour cells with the cyclopeptide YSNSG (SEQ ID NO: 3) inhibited tumour growth in a manner which was as effective as with the linear heptapeptide CNYYSNS (SEQ ID NO: 5) (FIG. 16).

In a complementary manner, one week after subcutaneous injection of B16F1 melanoma cells into the left flank of C57BI6 mice in an amount of 250000 cells in 0.1 ml of RPMI 1640 medium, the cyclopeptides of the invention were administered to various series of animals as follows:
a. peritoneal injection of cyclopeptides (at a concentration of 5 mg/kg weight of mouse) daily, alone or bound to biodegradable nanoparticles;
b. intravenous injection of cyclopeptides under the same conditions as option a);
c. per os administration: the cyclopeptides were mixed with food (in an amount of 10 mg/kg weight of mouse) and all administered using a gastric tube; and
d. subcutaneous injection of a solution of cyclopeptides contro-laterally with respect to injection of the cancer cells.

In all cases, the effect of the peptides was evaluated by measuring the kinetics of the tumour volume (percentage of tumour volume with treatment with respect to the tumour volume without treatment).

### B.10. Anti-Angiogenic Effect of YSNSG (SEQ ID NO: 3) Cyclopeptide

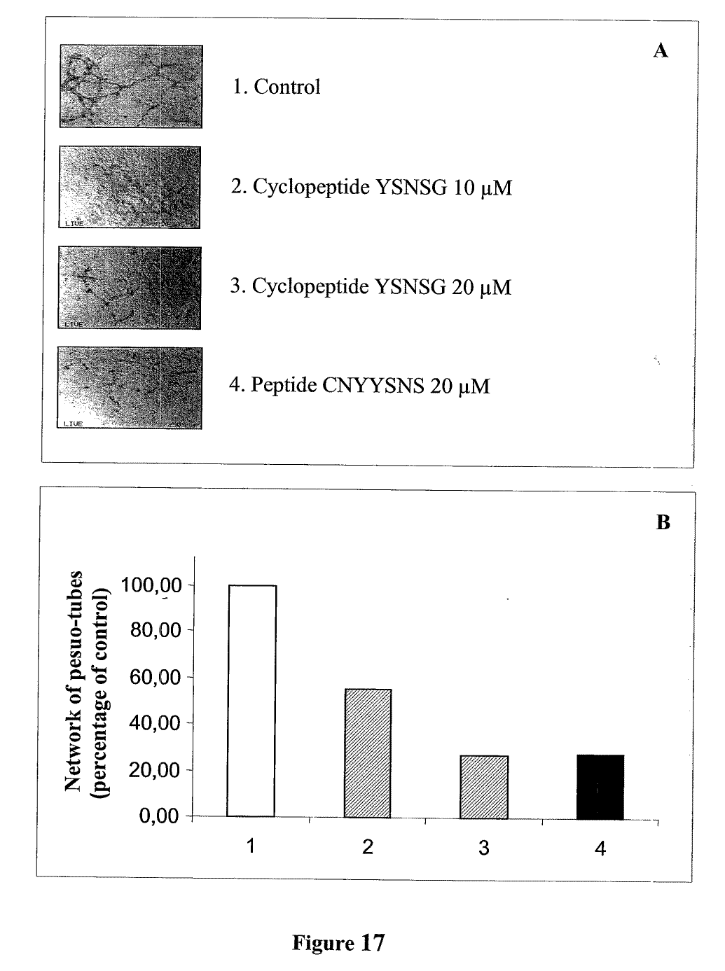

The anti-angiogenic activity was evaluated by the capacity of endothelial cells (HMEC-1 cells) to form capillary pseudotubes when they were cultivated on a Matrigel gel. As can be seen from the photographs in FIG. 17A, the addition of cyclopeptides (10 or 20 μM) strongly inhibits the formation of capillary pseudotubes in a manner which is comparable to the addition of the linear peptide CNYYSNS (SEQ ID NO: 5). Thus, the addition of 10 μM of YSNSG (SEQ ID NO: 3) cyclopeptide reduces the formation of pseudotubes by 40%, while the addition of 20 μM YSNSG (SEQ ID NO: 3) cyclopeptide reduces the formation by up to 70%, in a similar manner to the linear peptide (FIG. 17B).

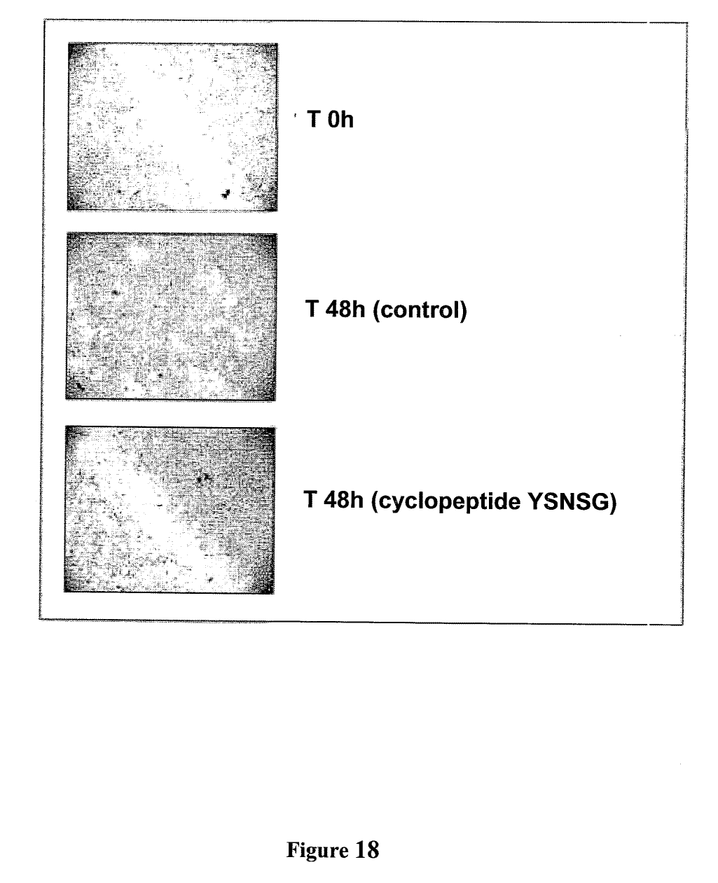

Similar results were obtained with HUVEC cells on which the cyclopeptide YSNSG (SEQ ID NO: 3) (20 μM) exerted a 45% inhibition. This inhibition could theoretically be exerted on the cell proliferation or cell migration. The cyclopeptide YSNSG (SEQ ID NO 3) inhibited 83% of the migration of the HUVEC cells (FIG. 18) after 48 h incubation in an artificial wound model. In contrast, the cyclopeptide YSNSG (SEQ ID NO: 3) did not modify cell proliferation.

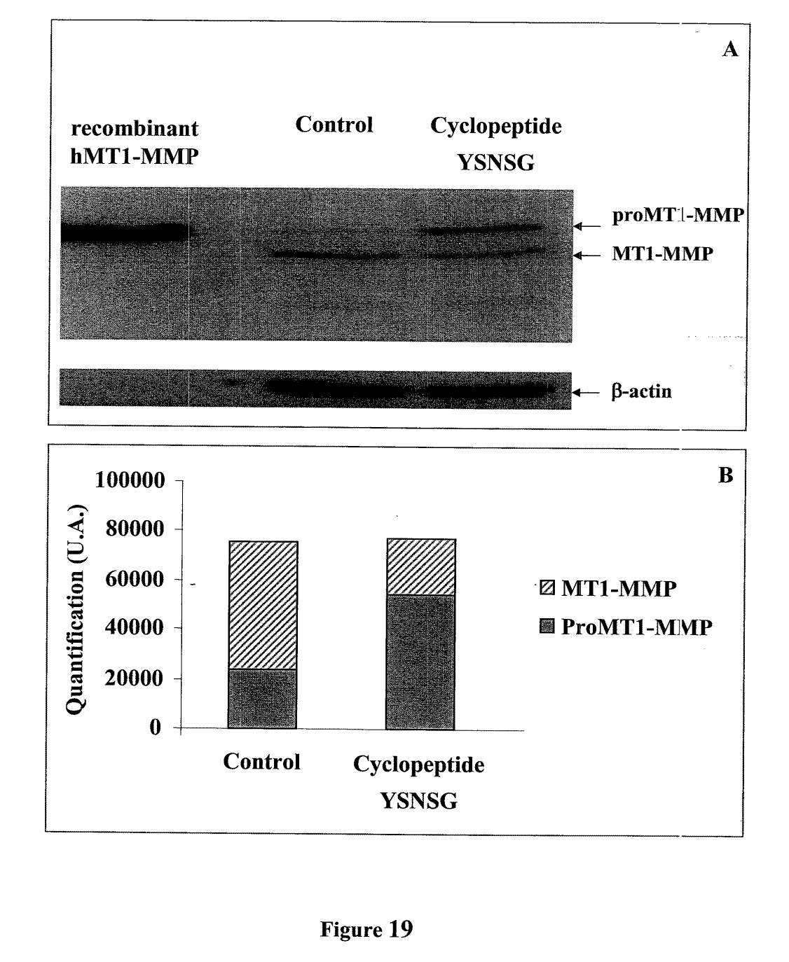

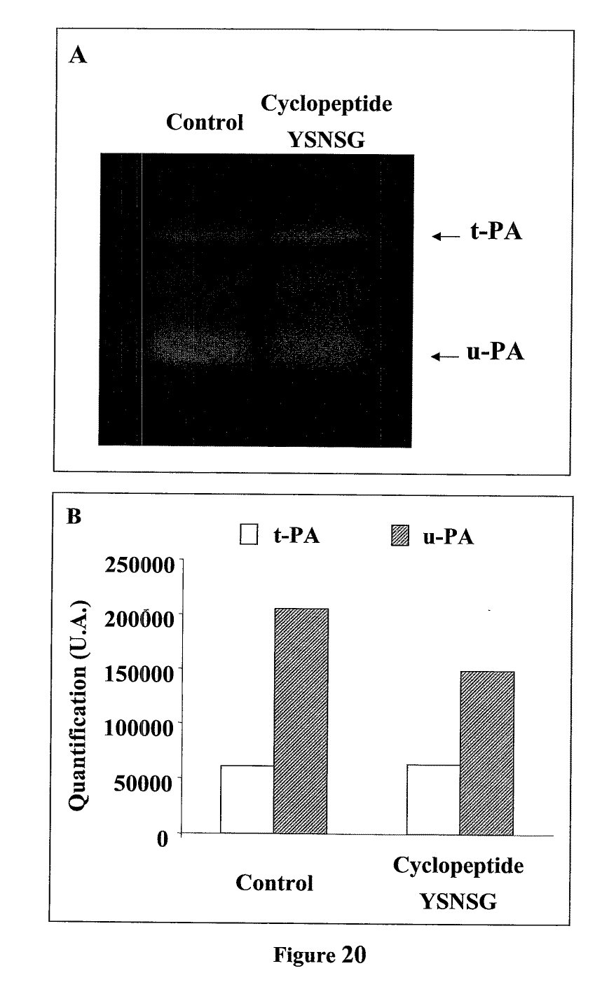

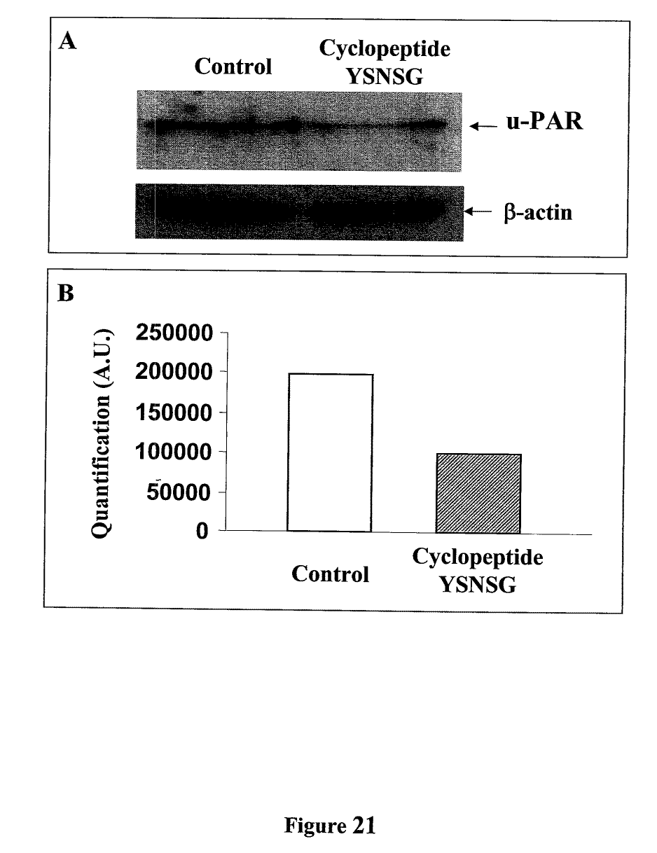

This inhibition in migration is primarily explained by a reduction of 56% in the activation of MT1-MMP, a matrix metalloproteinase capable of degrading the extracellular matrix and widely involved in angiogenesis (FIG. 19), and also by a reduction in the production of u-PA (−27%), a plasminogen activator, and its receptor, u-PAR (−49%), both also involved in the degradation of the extracellular matrix and cellular migration (FIGS. 20 and 21).

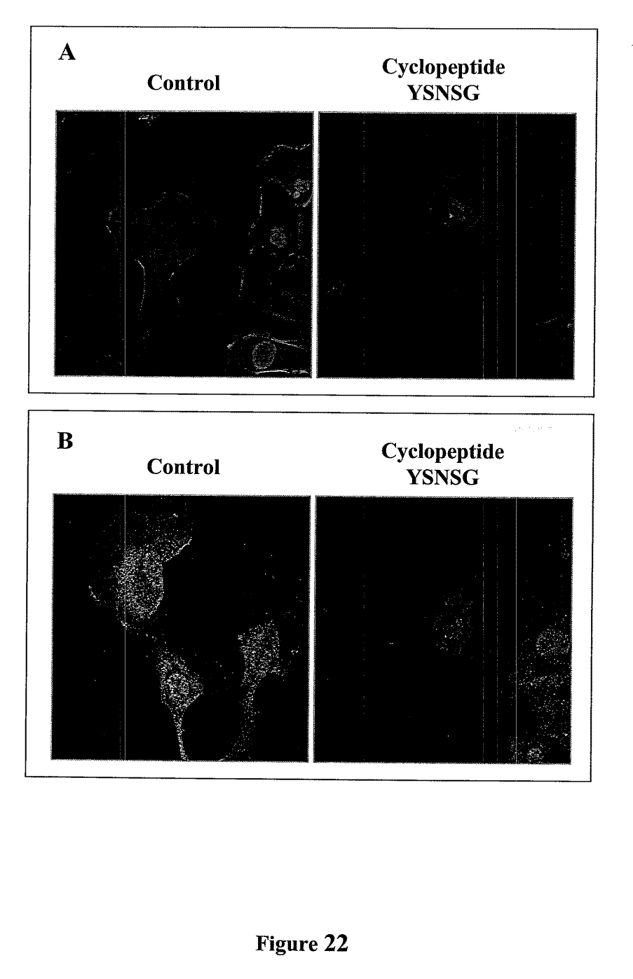

Inhibition of migration is manifested by a modification of the organization of the actin cytoskeleton and by a reduction in the expression of the phosphorylated form of focal adhesion kinase (phospho-FAK) (FIG. 22), which witnesses a reduction in migratory phenotype.

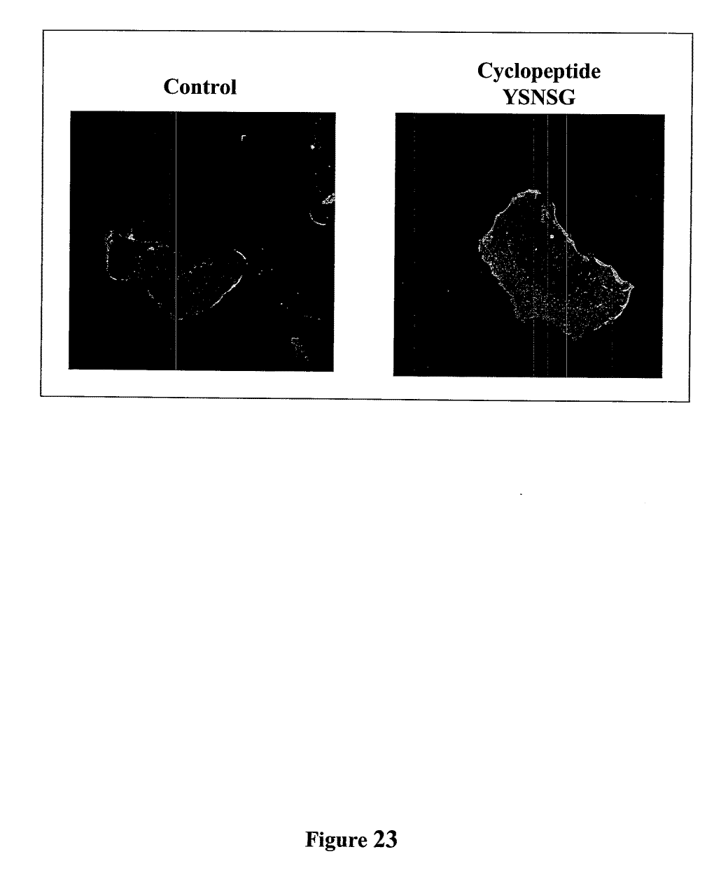

Expression of the β1 sub-unit of integrin at the cellular periphery is enhanced in the presence of cyclopeptide (FIG. 23), which reflects an increase in cellular anchoring points and a reduction in the migratory properties of the cell.

### Discussion

The biological activity of the cyclopentapeptide YSNSG (SEQ ID NO: 3) was compared with that of the natural peptide CNYYSNS (SEQ ID NO: 5). The cyclopeptide inhibits the in vitro proliferation of UACC-903 human melanoma cells as well as their invasive properties evaluated by their capacity to migrate through membranes covered with a reconstituted extracellular matrix, Matrigel (FIGS. 9 and 10). The intensity of the inhibition achieved is at least equal to that of the natural peptide, at the tested concentrations. This in vitro inhibition of invasive properties is manifested by a very large reduction in the expression, but above all in the activation, of MMP-14 (FIG. 12) associated with an increase in the secretion of the TIMP-2 inhibitor (FIG. 13). This inhibition is also exerted on the plasminogen activation cascade by reducing the secretion of u-PA and t-PA, which is as intense as that induced by the natural peptide CNYYSNS (SEQ ID NO: 5) (FIG. 14).

Similarly, the anti-cancer activity of the cyclopentapeptide YSNSG (SEQ ID NO: 3) was measured in the experimental in vivo mouse melanoma model. To this end, B16F1 mouse melanoma cells were treated with cyclopentapeptide then injected into the left flank of C57BL6 synergic mice. The cyclopentapeptide YSNSG (SEQ ID NO: 3) induced a reduction in tumour volume of 46% (FIG. 16). In the present case, only prior treatment of cells was used without re-injection of the peptide into the peri-tumoral region on days 7 and 14.

### LITERATURE

Brooks, B. R., Bruccoleri, R. E., Olafson, B. D., States, D J., Swaminathan, S., & Karplus, M. (1983) J. Comp. Chem. 4, 187-217.
Deryugina E I, Ratnikov B, Monosov E, Postnova T I, DiScipio R, Smith J W, et al. (2001) Exp. Cell Res., 263, 209-233.
Egeblad M, Werb Z. (2002) New functions for the matrix metalloproteinases in cancer progression. Nat. Rev. Cancer, 2, 161-173.
Essmann, U., Perera, L., Berkowitz, M. L., Darden, T., Lee, H., & Pedersen, L. G. (1995) J. Chem. Phys. 103, 8577.
Floquet N., Pasco S., Ramont L., Derreumaux P., Laronze J. Y., Nuzillard J. M., Maquart F. X. Alix A J., Monboisse J. C. (2004) J. Biol. Chem. 279(3): 2091-2100.
Han J, Ohno N, Pasco S, Monboisse J C, Borel J P, Kefalides N A. (1997) J. Biol. Chem., 272, 20395-20401.
Hornebeck W, Emonard H, Monboisse J C, Bellon G. (2002) Sem. Cancer Biol., 439, 1-11.
Humphrey, W., Dalke, A., & Schulten, K. (1996) J. Mol. Graph. 14, 33-38, 27-38.
Hutchinson E G, Thornton J M (1994) Protein Sci. December; 3(12):2207-16 Kalé, L., Skeel, R., Bhandarkar, M., Brunner, R., Gursoy, A., Krawetz, N., Phillips, J., Shinozaki, A., Varadarajan, K. & Schulten, K. (1999) Journal of Computational Physics. 151, 283-312.
Kalluri R. (2003) Nat. Rev. Cancer, 3, 422-433.
Kelley, L A, Gardner, S. P. & Sutcliffe, M J. (1996) Protein Eng. 9, 1063-1065.
MacKerell, J., A. D., Bashford, D., Bellott, M., Dunbrack, R. L., Jr., Evanseck, J. D., Field, M. J., Fischer, S., Gao, J., Guo, H., Ha, S., et al. (1998) Journal of Physical Chemistry B 102, 3586-3616.
Maeshima V; Sudhakar A, Lively J C, Ueki K, Kharbanda S, Kahn C R, Sonenberg N, Hynes R O, Kalluri R. (2002) 295, 140-143.
Maquart F X; Pasco S, Ramont L, Hornebeck W; Monboisse J C. (2004) Crit. Rev. Oncol. Haematol. 49, 199-202.
Martinella-Catusse C, Polelfe M, Noël A, Gilles C, Dehan P, Munaut C, et al. (2001) Lab. Invest. 81, 167-175.
Ortega N, Werb Z. (2002) J. CeII ScL 11, 4201-4214.
Pasco S (2000a), Han J, Gillery P, Bellon G, Maquart F X, Borel J P, et al. Cancer Res. 60, 467-473.
Pasco S (2000b), Monboisse J C, Kieffer N. J. Biol. Chem. 275, 32999-33007.
Pasco S, Ramont L, Maquart F X, Monboisse J C. (2004) Crit. Rev. Oncol. Haematol. 49, 221-233.
Pasco S, Brassart B, Ramont L, Maquart F X, Monboisse J C. (2005) Cancer Detect Prev. 29, 260-266.
Pardi, A., Billeter, M., Jr., & Wuthrich, K. (1984) J. Mol. Biol. 180, 741-751.
Phillips, J. C, Braun, R., Wang, W., Gumbart, J., Tajkhorshid, E., Villa, E., Chipot, C, Skeel, R. D., Kale, L. & Schulten, K. (2005) J. Comp. Chem. 26, 1781-1802.
Piotto M., Saudek V and Sklenar V. (1992) J. Biomol. NMR, 1992, 2, 661.
Settor R E B. (1998) Am. J. Pathol. 153, 1347-1351.
Shahan S, Ohno N, Pasco S, Borel J P, Monboisse J C, Kefalides N A (1999) Connect. Tissue Res. 40, 221-232.
Shahan T A, Ziaie Z, Pasco S, Fawzi A, Bellon G, Monboisse J C, et al. (1999) Cancer Res. 59, 4584-4590.
Thern B, Rudolph J, Jung G. (2002) Angew Chem Int Ed Engl. July 2:41(13):2307-9 Wald, M., Olejar, T., Sebkova, V., Zadinova, M., Boubelik, M., & Pouckova, P. (2001) (Cancer. Chemother. Pharmacol. 47, S16-S22.

1. A cyclopeptide, characterized in that it comprises the sequence YSNS.

2. A cyclopeptide according to claim 1, wherein the cycle of the cyclopeptide comprises the sequence YSNS or is constituted by the sequence YSNS.

3. A cyclopeptide according to claim 1, wherein it is 4 or more amino acids and less than 10 amino acids in size, preferably it is 4 to 6 amino acids in size.

4. A cyclopeptide according to claim 1, wherein it is capable of binding to αVβ3 integrin.

5. A cyclopeptide according to claim 1, wherein it forms a β-bend at the amino acids YSNS.

6.-7. (canceled)

8. A cyclopeptide according to claim 1, which has anti-tumoral properties.

9. A cyclopeptide according to claim 1, wherein its amino acid sequence consists of YSNS.

10. A cyclopentapeptide according to claim 3 consisting of the sequence YSNSX, wherein said cyclopentapeptide is homodetic and represented by the following formula: 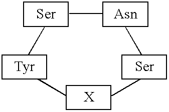
in which X is any amino acid that allows cyclisation of the same sequence-linear peptide.

11. A cyclopeptide according to claim 10, in which X is an amino acid with a small volume and/or an amino acid with a low charge or a neutral amino acid.

12. (canceled)

13. A cyclopeptide according to claim 11 wherein it is capable of binding αVβ3 integrin.

14. A cyclopentapeptide according to claim 1, the amino acid sequence of which consists of YSNSG, represented by the following formula:

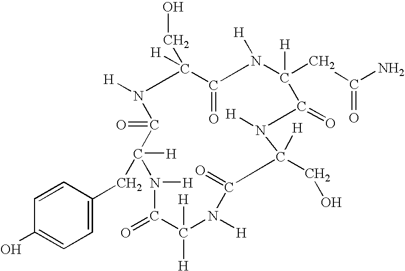

15. A cyclopeptide according to claim 14 wherein all the peptide bonds are in trans.

16. A cyclopeptide according to claim 15, which has the three-dimensional structure shown in FIG. 4.

17. (canceled)

18. A composition comprising a cyclopeptide according to claim 1.

19. A composition according to claim 18, further comprising a molecule which is biologically active in the treatment of cancer.

20. A composition according to claim 19, wherein the biologically active molecule is selected from at least one of the following molecules:
a. a chemotherapeutic agent;
b. a tumoral epitope specifically associated with tumour cells;
c. an antigen for cellular differentiation; and
d. interleukin 2 and/or interferon α.

21. (canceled)

22. Method for the treatment of cancer comprising administering in vivo a cyclopeptide according to claim 1.

23. Method according to claim 22, wherein the cancer is a cancer the tumour cells of which express the αVβ3 integrin molecule.

24. Method according to claim 23, wherein the cancer is melanoma, bronchial cancer, breast cancer or prostate cancer.

25. (canceled)

26. Method for inhibiting or reducing angiogenesis, preferably within tumours, comprising administering in vivo a cyclopeptide according to claim 1.

27. Method for reducing the proteolytic cascade associated with proMMP2 or the plasminogen activation system (u-PA), comprising administering in vivo a cyclopeptide according to claim 1.

28. (canceled)

29. (canceled)

30. Method according to claim 22 wherein the cyclopeptide is administered orally.

31. A kit which comprises a composition according to claim 18.

32. A kit according to claim 31, wherein the composition is formulated for parenteral, oral, subcutaneous or intravenous administration.

33. A composition comprising a cyclopeptide according to claim 10.

34. A composition comprising a cyclopeptide according to claim 14.


SEQUENCE LISTING (11 sequences)

SEQ ID NO: 1 - protein, 4 aa, Artificial Sequence
  Synthetic cyclopeptide
YSNS

SEQ ID NO: 2 - protein, 5 aa, Artificial Sequence
  Synthetic cyclopeptide
YSNSX

SEQ ID NO: 3 - protein, 5 aa, Artificial Sequence
  Synthetic cyclopeptide
YSNSG

SEQ ID NO: 4 - protein, 5 aa, Artificial Sequence
  Synthetic cyclopeptide
YSNSA

SEQ ID NO: 5 - protein, 7 aa, Artificial Sequence
  Synthetic linear heptapeptide
CNYYSNS

SEQ ID NO: 6 - protein, 19 aa, Artificial Sequence
  Chemically synthesized NC1 alpha3 (IV) 185-203
CNYYSNSYSFWLASLNPER

SEQ ID NO: 7 - protein, 5 aa, Artificial Sequence
  Synthetic cyclopeptide
SNSXY

SEQ ID NO: 8 - protein, 5 aa, Artificial Sequence
  Synthetic cyclopeptide
NSXYS

SEQ ID NO: 9 - protein, 5 aa, Artificial Sequence
  Synthetic cyclopeptide
SXYSN

SEQ ID NO: 10 - protein, 5 aa, Artificial Sequence
  Synthetic cyclopeptide
XYSNS

SEQ ID NO: 11 - protein, 10 aa, Artificial sequence
  Synthetic cyclopeptide
ACPYSNSSLC